(19) United States
(12) Patent Application Publication
(10) Pub. No.: US 2020/0172631 A1
(43) Pub. Date: Jun. 4, 2020

(54) ERBB2/HER2 MUTATIONS IN THE TRANSMEMBRANE OR JUXTAMEMBRANE DOMAIN

(71) Applicant: GENENTECH, INC., South San Francisco, CA (US)

(72) Inventor: Somasekar Seshagiri, South San Francisco, CA (US)

(73) Assignee: GENENTECH, INC., South San Francisco, CA (US)

(21) Appl. No.: 16/662,939

(22) Filed: Oct. 24, 2019

Related U.S. Application Data
(63) Continuation of application No. PCT/US2018/029116, filed on Apr. 24, 2018.
(60) Provisional application No. 62/489,382, filed on Apr. 24, 2017.
(60) Provisional application No. 62/560,564, filed on Sep. 19, 2017.

(51) Int. Cl. C07K 16/32; G01N 33/68; G01N 33/574; A61K 47/68

(52) U.S. Cl. CPC C07K 16/32; G01N 33/68; A61K 38/00; A61K 47/6803; G01N 33/574

(57) ABSTRACT

The present disclosure relates to somatic ErbB2 mutations in cancer and provides methods of identifying, diagnosing, and prognosing ErbB2-positive cancers. The present disclosure further provides methods of treating cancer, including certain subpopulations of patients. The mutations are in the transmembrane domain or juxtamembrane domain of ErbB2.

### RELATED APPLICATIONS

This application claims priority to U.S. Provisional Application Ser. No. 62/489,382, filed on Apr. 24, 2017, and U.S. Provisional Application Ser. No. 62/560,564, filed on Sep. 19, 2017, both of which are incorporated by reference in their entirety herein.

### FIELD OF THE INVENTION

The present disclosure relates to somatic ErbB2 (Her2) mutations in cancer and includes methods for the identification, diagnosis, prognosis of treatment outcome, and treatment of ErbB2 mutated cancers.

### BACKGROUND OF THE INVENTION

The human epidermal growth factor receptor (Her) family of receptor tyrosine kinases (RTK), also known as ErbB receptors, consists of four members: EGFR/ErbB1/Her1, ErbB2/Her2, ErbB3/Her3 and ErbB4/Her4 (Hynes et al. Nature Reviews Cancer 5, 341-354 (2005); Baselga et al. Nature Reviews Cancer 9, 463-475 (2009)). The ErbB family members contain an extracellular domain (ECD), a single-span transmembrane region, an intracellular tyrosine kinase domain, and a C-terminal signaling tail (Burgess et al. Mol Cell 12, 541-552 (2003); Ferguson. Annual Review of Biophysics 37, 353-373 (2008)). The ECD is a four domain structure consisting of two L domains (I and III) and two cysteine-rich domains (II and IV) (Burgess et al. Mol Cell 12, 541-552 (2003); Ferguson. Annual Review of Biophysics 37, 353-373 (2008)). The ErbB receptors are activated by multiple ligands that include epidermal growth factor (EGF), transforming growth factor-α (TGF-α) and neuregulins (Yarden et al. Nat Rev Mol Cell Biol 2, 127-137 (2001)). Activation of the receptor involves a single ligand molecule binding simultaneously to domains I and III, leading to heterodimerization or homodimerization through a dimerization arm in domain II (Burgess et al. Mol Cell 12, 541-552 (2003); Ogiso et al. Cell 110, 775-787 (2002); Cho. Science 297, 1330-1333 (2002); Dawson et al. Molecular and Cellular Biology 25, 7734-7742 (2005); Alvarado et al. Cell 142, 568-579 (2010); Lemmon et al. Cell 141, 1117-1134 (2010)). In the absence of ligand, the domain II dimerization arm is tucked away via an intramolecular interaction with domain IV, leading to a “tethered”, auto-inhibited configuration (Burgess et al. Mol Cell 12, 541-552 (2003); Cho. Science 297, 1330-1333 (2002); Lemmon et al. Cell 141, 1117-1134 (2010); Ferguson et al. Mol Cell 11, 507-517 (2003)).

Although the four ErbB receptors share a similar domain organization, functional and structural studies show that ErbB2 does not bind any of the known ErbB family ligands and is constitutively in an “untethered” (open) conformation suitable for dimerization (Garrett et al. Mol Cell 11, 495-505 (2003). In contrast, ErbB3, though capable of ligand binding, heterodimerization and signaling, has an impaired kinase domain (Baselga et al. Nature Reviews Cancer 9, 463-475 (2009); Jura et al. Proceedings of the National Academy of Sciences 106, 21608-21613 (2009); Shi et al. Proceedings of the National Academy of Sciences 107, 7692¬7697 (2010). Although, ErbB2 and ErbB3 are functionally incomplete on their own, their heterodimers are potent activators of cellular signaling (Pinkas-Kramarski et al. The EMBO Journal 15, 2452-2467 (1996); Tzahar et al. Molecular and Cellular Biology 16, 5276-5287 (1996); Holbro et al. Proceedings of the National Academy of Sciences 100, 8933-8938 (2003)).

While the ErbB receptors are critical regulators of normal growth and development, their deregulation has also been implicated in development and progression of cancers (Baselga et al. Nature Reviews Cancer 9, 463-475 (2009); Sithanandam et al. Cancer Gene Ther 15, 413-448 (2008); Hynes et al. Current Opinion in Cell Biology 21, 177-184 (2009)). In particular, gene amplification leading to receptor overexpression and activating somatic mutations are known to occur in ErbB2 and EGFR in various cancers (Sithanandam et al. Cancer Gene Ther 15, 413-448 (2008); Hynes et al. Current Opinion in Cell Biology 21, 177-184 (2009); Wang et al. Cancer Cell 10, 25-38 (2006); Yamauchi et al. Biomark Med 3, 139-151 (2009)). This has led to the development of multiple small molecule and antibody based therapeutics that target EGFR and ErbB2 (Baselga et al. Nature Reviews Cancer 9, 463-475 (2009); Alvarez et al. Journal of Clinical Oncology 28, 3366-3379 (2010)). Although the precise role of ErbB4 in oncogenesis is not well established (Koutras et al. Critical Reviews in Oncology/Hematology 74, 73-78 (2010)), transforming somatic mutations in ErbB4 have been reported in melanoma (Prickett et al. Nature Genetics 41, 1127-1132 (2009)).

Recently, ErbB2 (Her2) mutations have been shown to contribute to tumorigenesis (Bose et al., 2013). Such mutations have been described in the ECD and the kinase domain of ErbB2 (Bose et al., 2013; Chmielecki et al., 2015; Greulich et al., 2012; Wang et al., 2006). More recently, mutations in the transmembrane (TM) and juxtamembrane (JM) domains of Her2 have been reported in cancers (Ou et al., 2017; Yamamoto et al., 2014). The need exists to identify ErbB2 mutations that are predictive of response to Her2 targeting therapy.

### SUMMARY OF THE INVENTION

The present disclosure relates to ErbB2 (Her2) mutations that are present in cancer. The present disclosure further provides methods for identifying, diagnosing and prognosing ErbB2-positive cancers, and provides methods of treating cancer that have one or more mutations in ErbB2.

In one aspect, the present disclosure provides a method of treating cancer in a subject in need. In certain embodiments, the method comprises a) detecting in a biological sample obtained from the subject an ErbB2 somatic mutation in a nucleic acid sequence encoding ErbB2, wherein the mutation results in an amino acid variation at least one position within the transmembrane (TM) or juxtamembrane (JM) domain of a native human ErbB2 amino acid sequence and wherein the mutation is indicative of a cancer in the subject. In certain embodiments, the method further comprises b) administering an anti-cancer therapeutic agent to said subject. In certain embodiments, the mutation is an activating ErbB2 somatic mutation. In certain embodiments, the ErbB2 mutation is selected from the group of mutations listed in Table 1. In certain embodiments, the mutation is selected from the group consisting of V659E, G660D, G660R, R667Q, R678Q, Q709L and combinations thereof.

In another aspect, the present disclosure provides a method of treating an ErbB2-positive cancer in a subject that comprises a) detecting in a biological sample obtained from the subject the presence or absence of an amino acid mutation in the transmembrane (TM) or juxtamembrane (JM) domain of a native human ErbB2 amino acid sequence, wherein the ErbB2 mutation is selected from the group of mutations listed in Table 1, and wherein the presence of the mutation is indicative of a cancer in the subject. In certain embodiments, the method further comprises b) administering an anti-cancer therapeutic agent to said subject. In certain embodiments, the mutation is selected from the group consisting of V659E, G660D, G660R, R667Q, R678Q, Q709L and a combination thereof. In certain embodiments, the mutation is a Her2-activating mutation. In certain embodiments, the cancer is Her2-mutated. In certain embodiments, the cancer is selected from the group consisting of breast, gastric, colon, esophageal, rectal, cecum, colorectal, biliary, urothelial, bladder, salivary, non-small-cell lung (NSCLC) adenocarinoma, NSCLC (Squamous carcinoma), renal carcinoma, melanoma, ovarian, lung large cell, small-cell lung cancer (SCLC), hepatocellular (HCC), lung, and pancreatic. In a certain non-limiting embodiment, the cancer is breast cancer. In certain embodiments, the cancer is Her2/ErbB2-positive cancer. In certain embodiments, the cancer is considered a Her2/ErbB2-mutated cancer.

In certain embodiments, the methods of treatment involve administration of ErbB2 antagonists. In certain embodiments, the antagonist is a small molecule inhibitor. The small molecule inhibitory can be an ErbB2 kinase inhibitory small molecule drug. In certain non-limiting embodiments, the ErbB2 kinase inhibitory small molecule drug is lapatinib, afatinib or neratinib. In certain embodiments, the ErbB2 antagonist is an antagonist antibody. In certain embodiments, the antibody is selected from the group consisting of a monoclonal antibody, a bispecific antibody, a chimeric antibody, a human antibody, a humanized antibody and an antibody fragment. In certain embodiments, the ErbB2 antagonist is an antagonist anti-ErbB2 antibody or an anti-ErbB2 antibody-drug conjugate. In certain embodiments, the antibody is trastuzumab, trastuzumab-MCC-DM1 (T-DM1, trastuzumab emtansine), or pertuzumab.

The present disclosure further provides methods of determining the efficacy of an ErbB2 blocking antibody or antibody-drug conjugate. In certain embodiments, the method comprises a) detecting in a biological sample obtained from a subject treated with an ErbB2 blocking antibody a mutation in a nucleic acid sequence encoding ErbB2, wherein the mutation results in an amino acid variation at least one position within the transmembrane (TM) or juxtamembrane (JM) domain of a native human ErbB2 amino acid sequence, and wherein the mutation is indicative of an ErbB2 mutated cancer in the subject. In certain embodiments, the method further comprises b) predicting a therapeutic response in said subject based on the ErbB2 mutation detected. In certain embodiments, the ErB2 mutation is selected from the group of mutations listed in Table 1. In certain embodiments, the mutation is selected from the group consisting of V659E, G660D, G660R, R667Q, R678Q, Q709L and a combination thereof. In certain embodiments, the mutation is a Her2-activating mutation. In certain embodiments, the ErbB2 mutated cancer is selected from the group consisting of breast, gastric, colon, esophageal, rectal, cecum, colorectal, biliary, urothelial, bladder, salivary, non-small-cell lung (NSCLC) adenocarcinoma, NSCLC (Squamous carcinoma), renal carcinoma, melanoma, ovarian, lung large cell, small-cell lung cancer (SCLC), hepatocellular (HCC), lung, and pancreatic.

In certain embodiments, the methods of determining the efficacy of ErbB2 blocking antibodies involve ErbB2 antagonists. In certain embodiments, the antibody is selected from the group consisting of a monoclonal antibody, a bispecific antibody, a chimeric antibody, a human antibody, a humanized antibody and an antibody fragment. In certain embodiments, the antibody is trastuzumab, trastuzumab-MCC-DM1 (T-DM1), or pertuzumab. In certain embodiments the method comprises a) detecting in a biological sample obtained from a subject treated with an ErbB2 blocking antibody a mutation in a nucleic acid sequence encoding ErbB2, wherein the mutation results in an amino acid variation at least one position within the transmembrane (TM) or juxtamembrane (JM) domain of a native human ErbB2 amino acid sequence and wherein the mutation is indicative of an ErbB2 mutated cancer in the subject. In certain embodiments, the method further comprises predicting a therapeutic response in said subject based on the ErbB2 mutation detected. In certain embodiments, the ErB2 mutation is selected from the group of mutations listed in Table 1. In certain embodiments, the mutation is selected from the group consisting of V659E, G660D, G660R, R667Q, R678Q, Q709L and a combination thereof. In certain embodiments, the mutation is a Her2-activating mutation.

In another aspect, the present disclosure provides a method of treating a patient with an ErbB2-positive cancer which comprises a mutation in the TM region of the ErbB2 receptor. In certain embodiments, the method comprises administering to the patient an effective amount of trastuzumab or trastuzumab-MCC-DM1 (T-DM1). In certain embodiments, the mutation in the TM region is selected from the group of TM mutations provided in Table 1. In certain embodiments, the mutation in the TM region is at position V659 or G660. In certain embodiments, the mutation in the TM region is V659E, G660D or G660R.

In another aspect, the present disclosure provides a method of treating a patient with an ErbB2-positive cancer which comprises a mutation in the JM region of the ErbB2 receptor. In certain embodiments, the method comprises administering to the patient an effective amount of trastuzumab, trastuzumab-MCC-DM1 (T-DM1) or pertuzumab. In certain embodiments, the mutation in the JM region is selected from the group of JM mutations provided in Table 1. In certain embodiments, the mutation in the JM region is at position R667, R678 or Q709. In certain embodiments, the mutation in the JM region is R667Q, R678Q or Q709L. In certain embodiments, the ErbB2-positive cancer is selected from the group consisting of gastric, colon, esophageal, rectal, cecum, colorectal, non-small-cell lung (NSCLC) adenocarcinoma, NSCLC (Squamous carcinoma), renal carcinoma, melanoma, ovarian, lung large cell, small-cell lung cancer (SCLC), hepatocellular (HCC), lung, and pancreatic.

In another aspect, the present disclosure provides a method for diagnosing cancer in a subject. In certain embodiments, the method comprises detecting in a biological sample obtained from the subject a mutation in a nucleic acid sequence encoding ErbB2, wherein the mutation results in an amino acid variation at at least one position within the transmembrane (TM) or juxtamembrane (JM) domain of a native human ErbB2 amino acid sequence and wherein the mutation is indicative of an ErbB2 mutated cancer in the subject, and wherein the amino acid variation is selected from the group of mutations listed in Table 1 and indicates the presence of a cancer. In certain embodiments, the mutation is selected from the group consisting of V659E, G660D, G660R, R667Q, R678Q, Q709L and a combination thereof. In certain embodiments, the cancer is selected from the group consisting of breast, gastric, colon, esophageal, rectal, cecum, colorectal, biliary, urothelial, bladder, salivary, non-small-cell lung (NSCLC) adenocarcinoma, NSCLC (Squamous carcinoma), renal carcinoma, melanoma, ovarian, lung large cell, small-cell lung cancer (SCLC), hepatocellular (HCC), lung, and pancreatic.

In another aspect, the present disclosure provides a method for determining whether a patient is expected to be responsive to anti-ErbB2 therapy. In certain embodiments, the method comprises the steps of obtaining a sample of cellular material from a human subject; examining nucleic acid material from at least part of one or more ErbB2 genes in said cellular material; and determining whether such nucleic acid material comprises one or more mutations in a sequence encoding the transmembrane (TM) or juxtamembrane (JM) domain of a native human ErbB2 polypeptide. In certain embodiments, the ErbB2 mutation is selected from the group of mutations listed in Table 1.

In another aspect, the present disclosure provides a method for determining whether a patient is susceptible to therapy with trastuzumab or trastuzumab-MCC-DM1 (T-DM1). In certain embodiments, the method comprises the steps of determining whether the patient is suffering from an ErbB2 mutated cancer characterized by an amino acid mutation in the transmembrane (TM) domain of ErbB2; and administering trastuzumab or trastuzumab-MCC-DM1 (T-DM1) to patients with said ErbB2 mutated cancer. In certain embodiments, the mutation in the TM region is selected from the TM mutations provided in Table 1. In certain embodiments, the mutation in the TM region is at position V659 or G660. In certain embodiments, the mutation in the TM region is V659E, G660D or G660R.

In another aspect, the present disclosure provides method for determining whether a patient is susceptible to therapy with trastuzumab, trastuzumab-MCC-DM1 (T-DM1) or pertuzumab. In certain embodiments, the method comprises the steps of determining whether the patient is suffering from an ErbB2 mutated cancer characterized by an amino acid mutation in the juxtamembrane (JM) domain of ErbB2; and administering trastuzumab, trastuzumab-MCC-DM1 (T-DM1) or pertuzumab to patients with said ErbB2 mutated cancer. In certain embodiments, the mutation in the JM region is selected from the JM mutations provided in Table 1. In certain embodiments, the mutation in the JM region is at position R667, R678 or Q709. In certain embodiments, the mutation in the JM region is R667Q, R678Q, Q709L or a combination thereof. In certain embodiments, the ErbB2-positive cancer is selected from the group consisting of gastric, colon, esophageal, rectal, cecum, colorectal, non-small-cell lung (NSCLC) adenocarcinoma, NSCLC (Squamous carcinoma), renal carcinoma, melanoma, ovarian, lung large cell, small-cell lung cancer (SCLC), hepatocellular (HCC), lung, and pancreatic.

In yet another aspect, the present disclosure provides a method of improving the likelihood of response to treatment in a human patient with HER2-mutated cancer. In certain embodiments, the method comprises a) detecting in a biological sample obtained from the subject a mutation in a nucleic acid sequence encoding ErbB2, wherein the mutation results in an amino acid variation at at least one position within the transmembrane (TM) or juxtamembrane (JM) domain of a native human ErbB2 amino acid sequence and wherein the mutation is indicative of a cancer in the subject. In certain embodiments, the method further comprises b) administering trastuzumab, trastuzumab-MCC-DM1 (T-DM1) or pertuzumab to said subject. In certain embodiments, the ErB2 mutation is selected from the group of mutations listed in Table 1.

In certain embodiments, the present disclosure describes the use of an ErbB2 antagonist for the treatment of an ErbB2 mutated cancer characterized by an amino acid mutation in the transmembrane (TM) domain or juxtamembrane (JM) domain of ErbB2. In certain embodiments, the present disclosure describes the use of an ErbB2 antagonist to prepare a medicament for the treatment of an ErbB2 mutated cancer characterized by an amino acid mutation in the transmembrane (TM) domain or juxtamembrane (JM) domain of ErbB2. In certain embodiments, the mutation may be selected from the mutations listed in Table 1. In certain embodiments, the mutation can be selected from the group consisting of V659E, G660D, G660R, R667Q, R678Q and Q709L. In certain embodiments, the cancer can be selected from the group consisting of breast, gastric, colon, esophageal, rectal, cecum, colorectal, biliary, urothelial, bladder, salivary, non-small-cell lung (NSCLC) adenocarcinoma, NSCLC (Squamous carcinoma), renal carcinoma, melanoma, ovarian, lung large cell, small-cell lung cancer (SCLC), hepatocellular (HCC), lung, and pancreatic. In certain embodiments, the ErbB2 antagonist can be a small molecule inhibitor. In certain embodiments, the small molecule inhibitor may be an ErbB2 kinase inhibitor. In certain embodiments, the ErbB2 kinase inhibitor can be selected from the group consisting of lapatinib, afatinib and neratinib. In certain embodiments, the ErbB2 antagonist can be an antagonist anti-ErbB2 antibody or an anti-ErbB2 antibody-drug conjugate. In certain embodiments, the anti-ErbB2 antibody can be trastuzumab or pertuzumab. In certain embodiments, the ErbB2 antagonist can be trastuzumab-MCC-DM1 (T-DM1, trastuzumab emtansine).

### BRIEF DESCRIPTION OF THE DRAWINGS

FIG. 1 illustrates the BaF3 System used to assay the survival signaling by oncogenes.

FIG. 2 shows the level of cell survival signaling by Her2 mutants expressed in BaF3 in the presence and absence of wild-type Her2.

FIG. 3A-3C shows a workflow schematic of the saturation mutagenesis screen of the HER2 TM domain (A), a bar plot representing the allele frequency of HER2 mutations identified in the screen (B), and the allosteric mode of activation for the HER2 G660D mutant (C).

FIG. 4A-C demonstrate that V659E (A), G660D (B) and G660R (C) Her2 TM domain mutant mediated cell survival signaling is blocked by trastuzumab.

FIGS. 5A and 5B demonstrate that R667Q (A) and R678Q (B) Her2 JM domain mutant mediated cell survival signaling is blocked by trastuzumab and pertuzumab.

FIG. 6 demonstrates that Q709L JM domain mutant mediated survival signaling is blocked by transtuzumab and pertuzumab.

FIG. 7 demonstrates that Her2 TM/JM mutants respond to indicated ERBB2 kinase inhibitory small molecule drugs.

FIG. 8 shows schematics indicting the various domains of the ErbB2 receptor.

FIG. 9 shows the nucleic acid sequence of native human Her2/ErbB2 (Accession No. X03363) (SEQ ID NO: 1).

FIG. 10 shows the protein sequence of native human Her2/ErbB2 (Accession No. P04626) (SEQ ID NO: 2).

### DETAILED DESCRIPTION OF THE INVENTION

The practice of the present disclosure will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2nd edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4th edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction”, (Mullis et al., eds., 1994).

### Definitions

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 2nd. edition (1989) Cold Spring Harbor Laboratory Press, Cold Spring Harbor, N. Y. As appropriate, procedures involving the use of commercially available kits and reagents are generally carried out in accordance with manufacturer defined protocols and/or parameters unless otherwise noted. Before the present methods, kits and uses therefore are described, it is to be understood that this invention is not limited to the particular methodology, protocols, cell lines, animal species or genera, constructs, and reagents described as such may, of course, vary. It is also to be understood that the terminology used herein is for the purpose of describing particular embodiments only, and is not intended to limit the scope of the present disclosure which will be limited only by the appended claims.

It must be noted that as used herein and in the appended claims, the singular forms “a”, “and”, and “the” include plural referents unless the context clearly dictates otherwise.

Throughout this specification and claims, the word “comprise,” or variations such as “comprises” or “comprising,” will be understood to imply the inclusion of a stated integer or group of integers but not the exclusion of any other integer or group of integers.

The term “polynucleotide” or “nucleic acid,” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-2′-O-allyl, 2′-fluoro- or 2′-azido-ribose, carbocyclic sugar analogs, alpha-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR 2 (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Oligonucleotide,” as used herein, refers to short, single stranded polynucleotides that are at least about seven nucleotides in length and less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “primer” refers to a single stranded polynucleotide that is capable of hybridizing to a nucleic acid and allowing the polymerization of a complementary nucleic acid, generally by providing a free 3′-OH group.

As used herein, the term “gene” refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions as well as regulatory regions and can include 5′ and 3′ ends.

The term “somatic mutation” or “somatic variation” refers to a change in a nucleotide sequence (e.g., an insertion, deletion, inversion, or substitution of one or more nucleotides), which is acquired in a cell of the body as opposed to a germ line cell. The term also encompasses the corresponding change in the complement of the nucleotide sequence, unless otherwise indicated.

The term “activating mutation” or “activating somatic mutation” is used herein to refer to a mutation involved in driving tumorigenesis.

The term “amino acid variation” refers to a change in an amino acid sequence (e.g., an insertion, substitution, or deletion of one or more amino acids, such as an internal deletion or an N- or C-terminal truncation) relative to a reference sequence.

The term “variation” refers to either a nucleotide variation or an amino acid variation.

The term “a genetic variation at a nucleotide position corresponding to a somatic mutation,” “a nucleotide variation at a nucleotide position corresponding to a somatic mutation,” and grammatical variants thereof refer to a nucleotide variation in a polynucleotide sequence at the relative corresponding DNA position occupied by said somatic mutation. The term also encompasses the corresponding variation in the complement of the nucleotide sequence, unless otherwise indicated.

The term “array” or “microarray” refers to an ordered arrangement of hybridizable array elements, preferably polynucleotide probes (e.g., oligonucleotides), on a substrate. The substrate can be a solid substrate, such as a glass slide, or a semi-solid substrate, such as nitrocellulose membrane.

The term “amplification” refers to the process of producing one or more copies of a reference nucleic acid sequence or its complement. Amplification may be linear or exponential (e.g., the polymerase chain reaction (PCR)). A “copy” does not necessarily mean perfect sequence complementarity or identity relative to the template sequence. For example, copies can include nucleotide analogs such as deoxyinosine, intentional sequence alterations (such as sequence alterations introduced through a primer comprising a sequence that is hybridizable, but not fully complementary, to the template), and/or sequence errors that occur during amplification.

The term “mutation-specific oligonucleotide” refers to an oligonucleotide that hybridizes to a region of a target nucleic acid that comprises a nucleotide variation (often a substitution). “Somatic mutation-specific hybridization” means that, when a mutation-specific oligonucleotide is hybridized to its target nucleic acid, a nucleotide in the mutation-specific oligonucleotide specifically base pairs with the nucleotide variation. A somatic mutation-specific oligonucleotide capable of mutation-specific hybridization with respect to a particular nucleotide variation is said to be “specific for” that variation.

The term “target sequence,” “target nucleic acid,” or “target nucleic acid sequence” refers generally to a polynucleotide sequence of interest in which a nucleotide variation is suspected or known to reside, including copies of such target nucleic acid generated by amplification.

The term “detection” includes any means of detecting, including direct and indirect detection.

The terms “cancer” and “cancerous” refer to or describe the physiological condition in mammals that is typically characterized by unregulated cell growth. The cancer diagnosed and/or treated in accordance with the present disclosure is any type of cancer characterized by the presence of an ErbB2 mutation, specifically including metastatic or locally advanced non-resectable cancer, including, without limitation, breast cancer, squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, colon cancer, colorectal cancer, endometrial carcinoma, salivary gland carcinoma, kidney cancer, liver cancer, prostate cancer, vulval cancer, thyroid cancer, hepatic carcinoma and various types of head and neck cancer.

Herein, an “anti-cancer therapeutic agent” refers to a drug used to treat cancer. Non-limiting examples of anti-cancer therapeutic agents herein include chemotherapy agents, HER dimerization inhibitors, HER antibodies, HER antibody-drug conjugates, antibodies directed against tumor associated antigens, anti-hormonal compounds, cytokines, EGFR-targeted drugs, anti-angiogenic agents, tyrosine kinase inhibitors, growth inhibitory agents and antibodies, cytotoxic agents, antibodies that induce apoptosis, COX inhibitors, farnesyl transferase inhibitors, antibodies that binds oncofetal protein CA 125, HER2 vaccines, Raf or ras inhibitors, liposomal doxorubicin, topotecan, taxane, dual tyrosine kinase inhibitors, TLK286, EMD-7200, pertuzumab, trastuzumab, trastuzumab-MCC-DM1, erlotinib, and bevacizumab.

The term “ErbB2-positive cancer” or “Her2-positive cancer” refers to a cancer comprising cells which have Her2 protein present in the cells, e.g., at their cell surface. Her2 protein may be overexpressed, e.g., by gene amplification. Tumors overexpressing Her2 may be rated by immunohistochemical scores according to the number of copies of Her2 molecules expressed per cell, and can been determined biochemically: 0=0-10,000 copies/cell, 1+=at least about 200,000 copies/cell, 2+=at least about 500,000 copies/cell, 3+=at least about 2,000,000 copies/cell. Overexpression of Her2 at the 3+ level, which leads to ligand-independent activation of the tyrosine kinase (Hudziak et al., Proc. Natl. Acad. Sci. USA 84: 7159-7163 [1987]), occurs in approximately 30% of breast cancers, and in these patients, relapse-free survival and overall survival are diminished (Slamon et al., Science 244: 707-712 [1989]; Slamon et al., Science 235: 177-182 [1987]).

The term “ErbB2-mutated cancer” is used herein to refer to a cancer defined by an amino acid variation within the transmembrane (TM) domain or juxtamembrane (JM) domain of the ErbB2 amino acid sequence, especially the native human ErbB2 amino acid sequence of SEQ ID NO: 2.

“Early-stage breast cancer” or “early breast cancer” or “eBC”, as used herein, refers to breast cancer that has not spread beyond the breast or the axillary lymph nodes. Such cancer is generally treated with neoadjuvant or adjuvant therapy.

An “advanced” cancer is one which has spread outside the site or organ of origin, either by local invasion or metastasis. Accordingly, the term “advanced” cancer includes both locally advanced and metastatic disease, such as “advanced breast cancer”.

A “refractory” cancer is one which progresses even though an anti-tumor agent, such as a chemotherapy, is being administered to the cancer patient. An example of a refractory cancer is one which is platinum refractory.

A “recurrent” cancer is one which has regrown, either at the initial site or at a distant site, after a response to initial therapy, such as surgery.

A “locally recurrent” cancer is cancer that returns after treatment in the same place as a previously treated cancer.

A “non-resectable” or “unresectable” cancer is not able to be removed (resected) by surgery.

“Adjuvant therapy” or “adjuvant treatment” or “adjuvant administration” refers to systemic therapy given after surgery.

“Neoadjuvant therapy” or “neoadjuvant treatment” or “neoadjuvant administration” refers to systemic therapy given prior to surgery.

“Metastatic” cancer refers to cancer which has spread from one part of the body (e.g. the breast) to another part of the body.

As used herein, a subject “at risk” of developing cancer may or may not have detectable disease or symptoms of disease, and may or may not have displayed detectable disease or symptoms of disease prior to the diagnostic methods described herein. “At risk” denotes that a subject has one or more risk factors, which are measurable parameters that correlate with development of cancer, as described herein and known in the art. A subject having one or more of these risk factors has a higher probability of developing cancer than a subject without one or more of these risk factor(s).

The term “diagnosis” is used herein to refer to the identification or classification of a molecular or pathological state, disease or condition, for example, cancer. “Diagnosis” may also refer to the classification of a particular sub-type of cancer, e.g., by molecular features (e.g., a patient subpopulation characterized by nucleotide variation(s) in a particular gene or nucleic acid region).

The term “aiding diagnosis” is used herein to refer to methods that assist in making a clinical determination regarding the presence, or nature, of a particular type of symptom or condition of cancer. For example, a method of aiding diagnosis of cancer can comprise measuring the presence of absence of one or more genetic markers indicative of cancer or an increased risk of having cancer in a biological sample from an individual.

The term “prognosis” is used herein to refer to the prediction of the likelihood of developing cancer. The term “prediction” is used herein to refer to the likelihood that a patient will respond either favorably or unfavorably to a drug or set of drugs. In certain embodiments, the prediction relates to the extent of those responses. In certain embodiments, the prediction relates to whether and/or the probability that a patient will survive or improve following treatment, for example treatment with a particular therapeutic agent, and for a certain period of time without disease recurrence. The predictive methods of the present disclosure can be used clinically to make treatment decisions by choosing the most appropriate treatment modalities for any particular patient. The predictive methods of the present disclosure are valuable tools in predicting if a patient is likely to respond favorably to a treatment regimen, such as a given therapeutic regimen, including for example, administration of a given therapeutic agent or combination, surgical intervention, steroid treatment, etc., or whether long-term survival of the patient, following a therapeutic regimen is likely.

As used herein, “treatment” refers to clinical intervention in an attempt to alter the natural course of the individual or cell being treated, and can be performed before or during the course of clinical pathology. Desirable effects of treatment include preventing the occurrence or recurrence of a disease or a condition or symptom thereof, alleviating a condition or symptom of the disease, diminishing any direct or indirect pathological consequences of the disease, decreasing the rate of disease progression, ameliorating or palliating the disease state, and achieving remission or improved prognosis. In certain embodiments, methods and compositions of the present disclosure are useful in attempts to delay development of a disease or disorder.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

An “effective amount” refers to an amount effective, at dosages and for periods of time necessary, to achieve the desired therapeutic or prophylactic result. A “therapeutically effective amount” of a therapeutic agent may vary according to factors such as the disease state, age, sex, and weight of the individual, and the ability of the antibody to elicit a desired response in the individual. A therapeutically effective amount is also one in which any toxic or detrimental effects of the therapeutic agent are outweighed by the therapeutically beneficial effects. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. The effective amount may, for example, extend progression free survival (e.g. as measured by Response Evaluation Criteria for Solid Tumors, RECIST, or CA-125 changes), result in an objective response (including a partial response, PR, or complete response, CR), increase overall survival time, and/or improve one or more symptoms of cancer (e.g. as assessed by FOSI).

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount. An “individual,” “subject” or “patient” is a vertebrate. In certain embodiments, the vertebrate is a mammal. Mammals include, but are not limited to, primates (including human and non-human primates) and rodents (e.g., mice and rats). In certain embodiments, a mammal is a human.

A “patient subpopulation,” and grammatical variations thereof, as used herein, refers to a patient subset characterized as having one or more distinctive measurable and/or identifiable characteristics that distinguishes the patient subset from others in the broader disease category to which it belongs. Such characteristics include disease subcategories, gender, lifestyle, health history, organs/tissues involved, treatment history, etc. In certain embodiments, a patient subpopulation is characterized by nucleic acid signatures, including nucleotide variations in particular nucleotide positions and/or regions (such as somatic mutations).

A “control subject” refers to a healthy subject who has not been diagnosed as having cancer and who does not suffer from any sign or symptom associated with cancer.

The term “sample”, as used herein, refers to a composition that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized.

By “tissue or cell sample” is meant a collection of similar cells obtained from a tissue of a subject or patient. The source of the tissue or cell sample may be solid tissue as from a fresh, frozen and/or preserved organ or tissue sample or biopsy or aspirate; blood or any blood constituents; bodily fluids such as serum, urine, sputum, or saliva. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like. A “reference sample”, “reference cell”, “reference tissue”, “control sample”, “control cell”, or “control tissue”, as used herein, refers to a sample, cell or tissue obtained from a source known, or believed, not to be afflicted with the disease or condition for which a method or composition of the present disclosure is being used to identify. In certain embodiments, a reference sample, reference cell, reference tissue, control sample, control cell, or control tissue is obtained from a healthy part of the body of the same subject or patient in whom a disease or condition is being identified using a composition or method of the present disclosure. In certain embodiments, a reference sample, reference cell, reference tissue, control sample, control cell, or control tissue is obtained from a healthy part of the body of an individual who is not the subject or patient in whom a disease or condition is being identified using a composition or method of the present disclosure.

For the purposes herein a “section” of a tissue sample is a single part or piece of a tissue sample, e.g. a thin slice of tissue or cells cut from a tissue sample. It is understood that multiple sections of tissue samples may be taken and subjected to analysis according to the present disclosure, provided that it is understood that the present disclosure comprises a method whereby the same section of tissue sample is analyzed at both morphological and molecular levels, or is analyzed with respect to both protein and nucleic acid.

The terms “correlate” or “correlating” refer to the comparison, in any way, of the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the embodiment of gene expression analysis or protocol, one may use the results of the gene expression analysis or protocol to determine whether a specific therapeutic regimen should be performed.

A “small molecule” or “small organic molecule” is defined herein as an organic molecule having a molecular weight below about 500 Daltons.

The word “label,” used herein, refers to a detectable compound or composition. The label may be detectable by itself (e.g., radioisotope labels or fluorescent labels) or, in the case of an enzymatic label, may catalyze chemical alteration of a substrate compound or composition which results in a detectable product. Radionuclides that can serve as detectable labels include, for example, 1-131, 1-123, 1-125, Y-90, Re-188, Re-186, At-211, Cu-67, Bi-212, and Pd-109.

Reference to “about” a value or parameter herein includes (and describes) embodiments that are directed to that value or parameter per se. For example, description referring to “about X” includes description of “X.”

The term “package insert” is used herein to refer to instructions customarily included in commercial packages of therapeutic products, that contain information about the indications, usage, dosage, administration, combination therapy, contraindications and/or warnings concerning the use of such therapeutic products.

The terms “antibody” and “immunoglobulin” are used interchangeably in the broadest sense and include monoclonal antibodies (e.g., full length or intact monoclonal antibodies), polyclonal antibodies, monovalent antibodies, multivalent antibodies, multispecific antibodies (e.g., bispecific antibodies so long as they exhibit the desired biological activity) and may also include certain antibody fragments (as described in greater detail herein). An antibody can be chimeric, human, humanized and/or affinity matured. “Antibody fragments” comprise a portion of an intact antibody, preferably comprising the antigen binding region thereof. Examples of antibody fragments include Fab, Fab′, F(ab′)2, and Fv fragments; diabodies; linear antibodies; single-chain antibody molecules; and multispecific antibodies formed from antibody fragments.

An antibody of the present disclosure “which binds” an antigen of interest is one that binds the antigen with sufficient affinity such that the antibody is useful as a diagnostic and/or therapeutic agent in targeting a protein or a cell or tissue expressing the antigen. With regard to the binding of an antibody to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target.

A “Her receptor” or “ErbB receptor” is a receptor protein tyrosine kinase which belongs to the Her receptor family and includes EGFR (ErbB 1, Her1), Her2 (ErbB2), Her3 (ErbB3) and Her4 (ErbB4) receptors.

The terms “ErbB1”, “Her1”, “epidermal growth factor receptor” and “EGFR” are used interchangeably herein and refer to EGFR as disclosed, for example, in Carpenter et al Ann. Rev. Biochem. 56:881-914 (1987), including naturally occurring mutant forms thereof (e.g. a deletion mutant EGFR as in Ullrich et al, Nature (1984) 309:418425 and Humphrey et al. PNAS (USA) 87:4207-4211 (1990)), as well we variants thereof, such as EGFRvIII. Variants of EGFR also include deletional, substitutional and insertional variants, for example those described in Lynch et al (New England Journal of Medicine 2004, 350:2129), Paez et al (Science 2004, 304:1497), and Pao et al (PNAS 2004, 101:13306).

The expressions “ErbB2” and “Her2” are used interchangeably herein and refer to human Her2 protein described, for example, in Semba et al, PNAS (USA) 82:6497-6501 (1985) and Yamamoto et al. Nature 319:230-234 (1986) (GenBank accession number X03363). In certain embodiments, ErbB2 receptor comprises the amino acid sequence shown in SEQ ID NO: 2.

As used herein, “Her2 extracellular domain” or “Her2 ECD” refers to a domain of Her2 that is outside of a cell, either anchored to a cell membrane, or in circulation, including fragments thereof. In certain embodiments, the extracellular domain of Her2 may comprise four domains: “Domain I” (amino acid residues from about 22-195, “Domain II” (amino acid residues from about 196-321), “Domain III” (amino acid residues from about 322-498), and “Domain IV” (amino acid residues from about 499-648) (residue numbering without signal peptide). See Garrett et al. Mol. Cell. 11:495-505 (2003), Cho et al. Nature 421:756-760 (2003), Franklin et al. Cancer Cell 5:317-328 (2004), and Plowman et al. Proc. Natl. Acad. Sci 90:1746-1750 (1993).

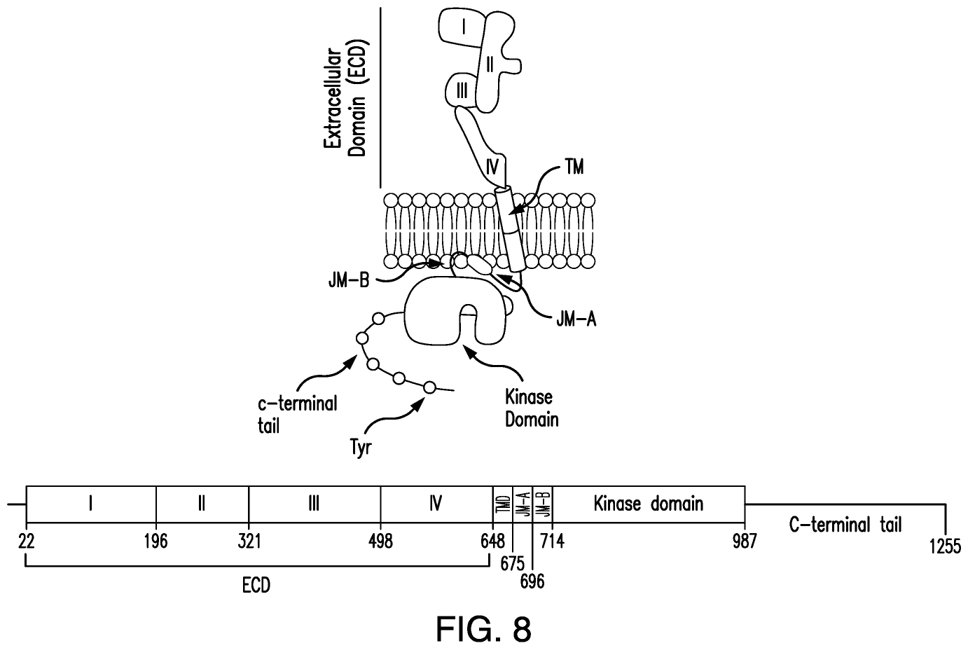

The Her2 “transmembrane domain” or “TM domain” refers to a segment of the protein that spans the entire phospholipid bilayer of the cell membrane and which has three-dimensional structure that is thermodynamically stable in a membrane. This may be, for example, a single alpha helix, a transmembrane beta barrel, or a beta-helix structure that is typically composed of more hydrophobic residues. In certain embodiments, the transmembrane domain of Her2 comprises amino acid residues from about 649-675 (see FIG. 8).

The Her2 “juxtamembrane domain” or “JM domain” refers to a domain that connects the transmembrane domain with the catalytic domain, and likely works synergistically with the TM domain in signal transduction. The juxtamembrane domain is usually of 40-80 residues long, and contains several basic residues (Lys and Arg) located close to the membrane surface. Amino acids in this region have been shown to serve as binding and phosphorylation sites for signaling molecules. In certain embodiments, the transmembrane domain of Her2 comprises amino acid residues from about 676-714 (see FIG. 8).

“ErbB3” and “Her3” refer to the receptor polypeptide as disclosed, for example, in U.S. Pat. Nos. 5,183,884 and 5,480,968 as well as Kraus et al. PNAS (USA) 86:9193-9197 (1989).

Herein, “Her3 extracellular domain” or “Her3 ECD” or “ErbB3 extracellular domain” refers to a domain of Her3 that is outside of a cell, either anchored to a cell membrane, or in circulation, including fragments thereof.

The terms “ErbB4” and “Her4” herein refer to the receptor polypeptide as disclosed, for example, in EP Pat Appl. No 599,274; Plowman et al., Proc. Natl. Acad. Sci USA, 90:1746-1750 (1993); and Plowman et al., Nature, 366:473-475 (1993), including isoforms thereof, e.g., as disclosed in WO99/19488, published Apr. 22, 1999. The term “epitope” refers to the particular site on an antigen molecule to which an antibody binds.

The “epitope 4 D5” or “4 D5 epitope” or “4 D5” is the region in the extracellular domain of Her2 to which the antibody 4 D5 (ATCC CRL 10463) and trastuzumab bind. This epitope is close to the transmembrane domain of Her2, and within domain IV of Her2. To screen for antibodies which bind to the 4 D5 epitope, a routine cross-blocking assay such as that described in Antibodies, A Laboratory Manual, Cold Spring Harbor Laboratory, Ed Harlow and David Lane (1988), can be performed. Alternatively, epitope mapping can be performed to assess whether the antibody binds to the 4 D5 epitope of Her2 (e.g. any one or more residues in the region from about residue 550 to about residue 610, inclusive, of Her2 (SEQ ID NO: 2).

The “epitope 2C4” or “2C4 epitope” is the region in the extracellular domain of Her2 to which the antibody 2C4 binds. In order to screen for antibodies which bind to the 2C4 epitope, a routine cross-blocking assay such as that described in Antibodies, A Laboratory Manual, Cold Spring Harbor Laboratory, Ed Harlow and David Lane (1988), can be performed. Alternatively, epitope mapping can be performed to assess whether the antibody binds to the 2C4 epitope of Her2. Epitope 2C4 comprises residues from domain II in the extracellular domain of Her2. The 2C4 antibody and pertuzumab bind to the extracellular domain of Her2 at the junction of domains I, II and III (Franklin et al. Cancer Cell 5:317-328 (2004)).

A “Her heterodimer” herein is a noncovalently associated heterodimer comprising at least two different Her receptors, such as EGFR-Her2, EGFR-Her3, EGFR-Her4, Her2-Her3 or Her2-Her4 heterodimers.

A “Her inhibitor” or “ErbB inhibitor” or “ErbB antagonist” is an agent which interferes with Her activation or function. Examples of Her inhibitors include Her antibodies (e.g. EGFR, Her2, Her3, or Her4 antibodies); EGFR-targeted drugs; small molecule Her antagonists; Her tyrosine kinase inhibitors; Her2 and EGFR dual tyrosine kinase inhibitors such as lapatinib/GW572016; antisense molecules (see, for example, WO 2004/87207); and/or agents that bind to, or interfere with function of, downstream signaling molecules, such as MAPK or Akt. Preferably, the Her inhibitor is an antibody which binds to a Her receptor. In general, a Her inhibitor refers to those compounds that specifically bind to a particular Her receptor and prevent or reduce its signaling activity, but do not specifically bind to other Her receptors. For example, a Her3 antagonist specifically binds to reduce its activity, but does not specifically bind to EGFR, Her2, or Her4.

A “Her dimerization inhibitor” or “HDI” is an agent which inhibits formation of a Her homodimer or Her heterodimer. Preferably, the Her dimerization inhibitor is an antibody. However, Her dimerization inhibitors also include peptide and non-peptide small molecules, and other chemical entities which inhibit the formation of Her homo- or heterodimers.

An antibody which “inhibits Her dimerization” is an antibody which inhibits, or interferes with, formation of a Her dimer, regardless of the underlying mechanism. In certain embodiments, such an antibody binds to Her2 at the heterodimeric binding site thereof. One particular example of a dimerization inhibiting antibody is pertuzumab (Pmab), or MAb 2C4. Other non-limiting examples of Her dimerization inhibitors include antibodies which bind to EGFR and inhibit dimerization thereof with one or more other Her receptors (for example EGFR monoclonal antibody 806, MAb 806, which binds to activated or “untethered” EGFR; see Johns et al, J. Biol. Chem. 279(29):30375-30384 (2004)); antibodies which bind to Her3 and inhibit dimerization thereof with one or more other Her receptors; antibodies which bind to Her4 and inhibit dimerization thereof with one or more other Her receptors; peptide dimerization inhibitors (U.S. Pat. No. 6,417,168); antisense dimerization inhibitors; etc.

A “Her antibody” is an antibody that binds to a Her receptor. Optionally, the Her antibody further interferes with Her activation or function. Particular Her2 antibodies include pertuzumab and trastuzumab. Examples of particular EGFR antibodies include cetuximab and panitumumab. Patent Publications related to Her2 antibodies include: U.S. Pat. Nos. 5,677,171; 5,720,937; 5,720,954; 5,725,856; 5,770,195; 5,772,997; 6,165,464; 6,387,371; 6,399,063; 6,015,567; 6,333,169; 4,968,603; 5,821,337; 6,054,297; 6,407,213; 6,639,055; 6,719,971; 6,800,738; 5,648,237; 7,018,809; 6,267,958; 6,695,940; 6,821,515; 7,060,268; 7,682,609; 7,371,376; 6,127,526; 6,333,398; 6,797,814; 6,339,142; 6,417,335; 6,489,447; 7,074,404; 7,531,645; 7,846,441; 7,892,549; 6,573,043; 6,905,830; 7,129,840; 7,344,840; 7,468,252; 7,674,589; 6,949,245; 7,485,302; 7,498,030; 7,501,122; 7,537,931; 7,618,631; 7,862,817; 7,041,292; 6,627,196; 7,371,379; 6,632,979; 7,097,840; 7,575,748; 6,984,494; 7,279,287; 7,811,773; 7,993,834; 7,435,797; 7,850,966; 7,485,704; 7,807,799; 7,560,111; 7,879,325; 7,449,184; 7,700,299; 8,591,897; and US 2010/0016556; US 2005/0244929; US 2001/0014326; US 2003/0202972; US 2006/0099201; US 2010/0158899; US 2011/0236383; US 2011/0033460; US 2005/0063972; US 2006/018739; US 2009/0220492; US 2003/0147884; US 2004/0037823; US 2005/0002928; US 2007/0292419; US 2008/0187533; US 2003/0152987; US 2005/0100944; US 2006/0183150; US2008/0050748; US 2010/0120053; US 2005/0244417; US 2007/0026001; US 2008/0160026; US 2008/0241146; US 2005/0208043; US 2005/0238640; US 2006/0034842; US 2006/0073143; US 2006/0193854; US 2006/0198843; US 2011/0129464; US 2007/0184055; US 2007/0269429; US 2008/0050373; US 2006/0083739; US 2009/0087432; US 2006/0210561; US 2002/0035736; US 2002/0001587; US 2008/0226659; US 2002/0090662; US 2006/0046270; US 2008/0108096; US 007/0166753; US 2008/0112958; US 2009/0239236; US 2004/008204; US 2009/0187007; US 2004/0106161; US 2011/0117096; US 2004/048525; US 2004/0258685; US 2009/0148401; US 2011/0117097; US 2006/0034840; US 2011/0064737; US 2005/0276812; US 2008/0171040; US 2009/0202536; US 2006/0013819; US 2006/0018899; US 2009/0285837; US 2011/0117097; US 2006/0088523; US 2010/0015157; US 2006/0121044; US 2008/0317753; US2006/0165702; US 2009/0081223; US 2006/0188509; US 2009/0155259; US 2011/0165157; US 2006/0204505; US 2006/0212956; US 2006/0275305; US 2007/0009976; US 2007/0020261; US 2007/0037228; US 2010/0112603; US 2006/0067930; US 2007/0224203; US 2008/0038271; US 2008/0050385; 2010/0285010; US 2008/0102069; US 2010/0008975; US 2011/0027190; US 2010/0298156; US 2009/0098135; US 2009/0148435; US 2009/0202546; US 2009/0226455; US 2009/0317387; and US 2011/0044977. The contents of which are hereby incorporated by reference in their entireties.

“Her activation” refers to activation, or phosphorylation, of any one or more Her receptors. Generally, Her activation results in signal transduction (e.g. that caused by an intracellular kinase domain of a Her receptor phosphorylating tyrosine residues in the Her receptor or a substrate polypeptide). Her activation may be mediated by Her ligand binding to a Her dimer comprising the Her receptor of interest. Her ligand binding to a Her dimer may activate a kinase domain of one or more of the Her receptors in the dimer and thereby results in phosphorylation of tyrosine residues in one or more of the Her receptors and/or phosphorylation of tyrosine residues in additional substrate polypeptides(s), such as Akt or MAPK intracellular kinases.

“Phosphorylation” refers to the addition of one or more phosphate group(s) to a protein, such as a Her receptor, or substrate thereof.

A “heterodimeric binding site” on Her2, refers to a region in the extracellular domain of Her2 that contacts, or interfaces with, a region in the extracellular domain of EGFR, Her3 or Her4 upon formation of a dimer therewith. The region is found in Domain II of Her2. Franklin et al. Cancer Cell 5:317-328 (2004).

A Her2 antibody that “binds to a heterodimeric binding site” of Her2, binds to residues in domain II (and optionally also binds to residues in other of the domains of the Her2 extracellular domain, such as domains I and III), and can sterically hinder, at least to some extent, formation of a Her2-EGFR, Her2-Her3, or Her2-Her4 heterodimer. Franklin et al. Cancer Cell 5:317-328 (2004) characterize the Her2-pertuzumab crystal structure, deposited with the RC SB Protein Data Bank (ID Code IS78), illustrating an exemplary antibody that binds to the heterodimeric binding site of Her2. An antibody that “binds to domain II” of Her2 binds to residues in domain II and optionally residues in other domain(s) of Her2, such as domains I and III.

“Isolated,” when used to describe the various antibodies disclosed herein, means an antibody that has been identified and separated and/or recovered from a cell or cell culture from which it was expressed. Contaminant components of its natural environment are materials that would typically interfere with diagnostic or therapeutic uses for the polypeptide, and can include enzymes, hormones, and other proteinaceous or non-proteinaceous solutes. In preferred embodiments, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated antibody includes antibodies in situ within recombinant cells, because at least one component of the polypeptide natural environment will not be present. Ordinarily, however, isolated polypeptide will be prepared by at least one purification step.

An “ErbB2-positive cancer detecting agent” refers to an agent that is capable of detecting a mutation associated with an ErbB2-positive cancer within an ErbB2 nucleic acid sequence or amino acid sequence. Typically, the detecting agent comprises a reagent capable of specifically binding to an ErbB2 sequence. In a preferred embodiment, the reagent is capable of specifically binding to an ErbB2 mutation in an ErbB2 nucleic acid sequence. In certain embodiments, the polynucleotide is a probe comprising a nucleic acid sequence that specifically hybridizes to an ErbB2 sequence comprising a mutation. In certain embodiments, the detecting agent comprises a reagent capable of specifically binding to an ErbB2 amino acid sequence. In certain embodiments, the amino acid sequence comprises a mutation as described herein. The detecting agents may further comprise a label.

### ErbB2 Somatic Mutations

The present disclosure provides methods of detecting the presence or absence of ErbB2 somatic mutations associated with cancer in a sample from a subject. The present disclosure further provided methods of diagnosing and prognosing cancer by detecting the presence or absence of one or more of these somatic mutations in a sample from a subject, wherein the presence of the somatic mutation indicates that the subject has cancer. ErbB2 somatic mutations associated with cancer risk were identified using strategies including genome-wide association studies, modifier screens, and family-based screening.

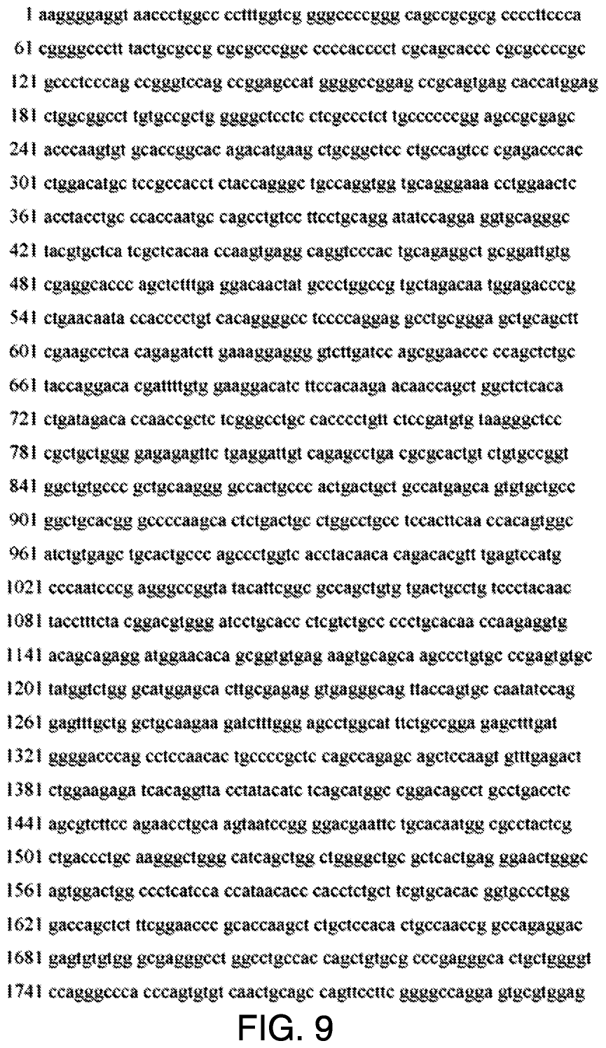

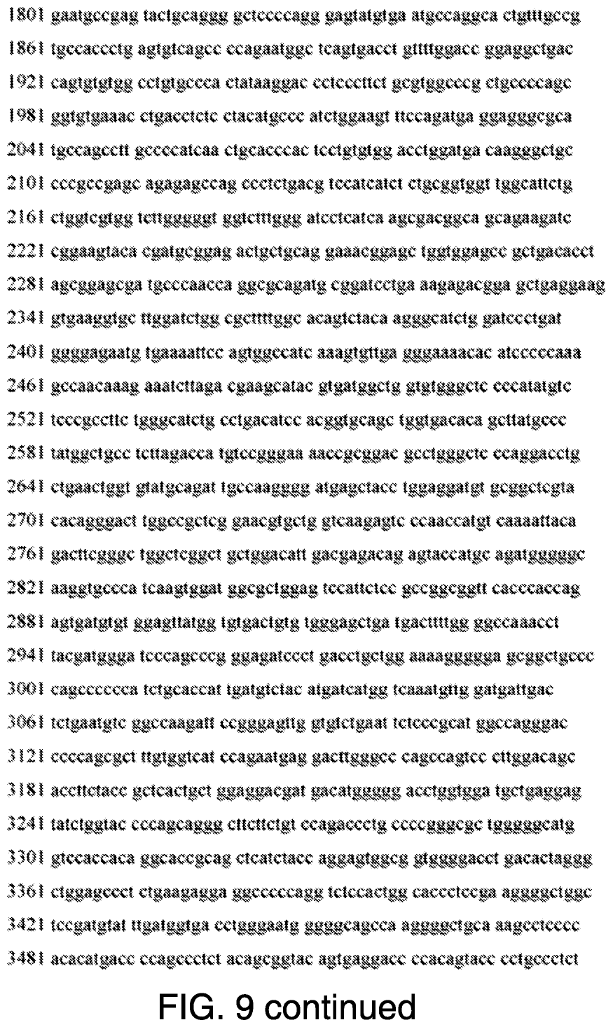

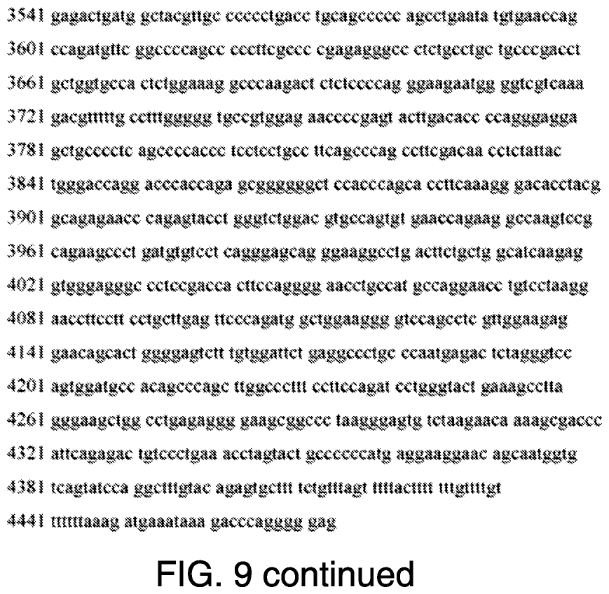

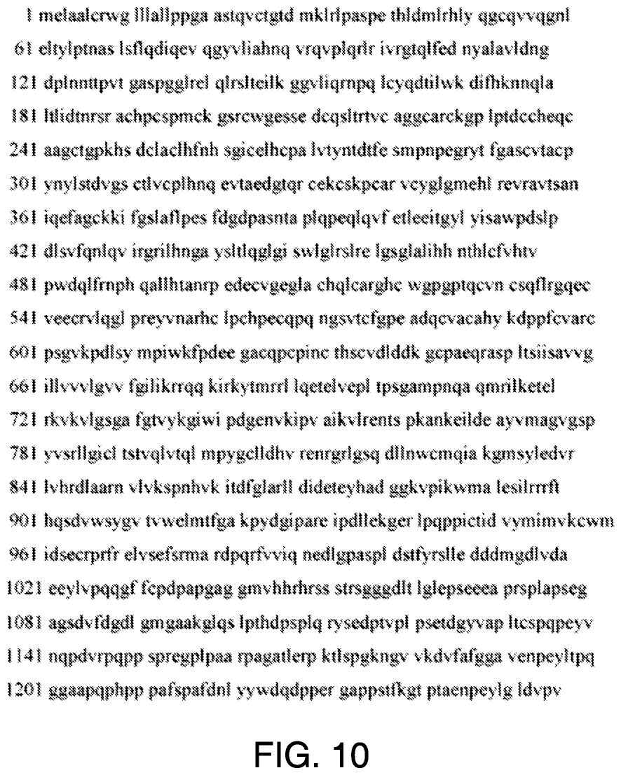

Somatic mutations or variations for use in the methods of the present disclosure include variations in ErbB2, or the genes encoding this protein. In certain embodiments, the somatic mutation is in genomic DNA that encodes a gene (or its regulatory region). In certain embodiments, the somatic mutation is a substitution, an insertion, or a deletion in a nucleic acid coding for ErbB2 (see nucleic acid sequence of SEQ ID NO: 1; FIG. 9 (Accession No. X03363); and protein sequence of SEQ ID NO:2, FIG. 10 (Accession No. P04626)). In certain embodiments, the variation is a mutation that results in an amino acid substitution in the transmembrane (TM), the juxtamembrane (JM) domain of Her2 and/or the regions adjacent. In certain embodiments, the variation is an amino acid substitution, insertion, truncation, or deletion in ErbB2. In certain embodiments, the variation is an amino acid substitution.

### Detection of Somatic Mutations

Nucleic acid, as used in any of the detection methods described herein, may be genomic DNA; RNA transcribed from genomic DNA; or cDNA generated from RNA. Nucleic acid may be derived from a vertebrate, e.g., a mammal. A nucleic acid is said to be “derived from” a particular source if it is obtained directly from that source or if it is a copy of a nucleic acid found in that source.

In certain embodiments, nucleic acid includes copies of the nucleic acid, e.g., copies that result from amplification. Amplification may be desirable in certain instances, e.g., in order to obtain a desired amount of material for detecting variations. The amplicons may then be subjected to a variation detection method, such as those described below, to determine whether a variation is present in the amplicon.

Somatic mutations or variations may be detected by certain methods known to those skilled in the art. Such methods include, but are not limited to, DNA sequencing; primer extension assays, including somatic mutation-specific nucleotide incorporation assays and somatic mutation-specific primer extension assays (e.g., somatic mutation-specific PCR, somatic mutation-specific ligation chain reaction (LCR), and gap-LCR); mutation-specific oligonucleotide hybridization assays (e.g., oligonucleotide ligation assays); cleavage protection assays in which protection from cleavage agents is used to detect mismatched bases in nucleic acid duplexes; analysis of MutS protein binding; electrophoretic analysis comparing the mobility of variant and wild type nucleic acid molecules; denaturing-gradient gel electrophoresis (DGGE, as in, e.g., Myers et al. (1985) Nature 313:495); analysis of RNase cleavage at mismatched base pairs; analysis of chemical or enzymatic cleavage of heteroduplex DNA; mass spectrometry (e.g., MALDI-TOF); genetic bit analysis (GBA); 5′ nuclease assays (e.g., TaqMan™); and assays employing molecular beacons. Certain of these methods are discussed in further detail below.

Detection of variations in target nucleic acids may be accomplished by molecular cloning and sequencing of the target nucleic acids using techniques well known in the art. Alternatively, amplification techniques such as the polymerase chain reaction (PCR) can be used to amplify target nucleic acid sequences directly from a genomic DNA preparation from tumor tissue. The nucleic acid sequence of the amplified sequences can then be determined and variations identified therefrom. Amplification techniques are well known in the art, e.g., the polymerase chain reaction is described in Saiki et al., Science 239:487, 1988; U.S. Pat. Nos. 4,683,203 and 4,683,195.

The ligase chain reaction, which is known in the art, can also be used to amplify target nucleic acid sequences. See, e.g., Wu et al., Genomics 4:560-569 (1989). In addition, a technique known as allele-specific PCR can also be used to detect somatic mutations (e.g., substitutions). See, e.g., Ruano and Kidd (1989) Nucleic Acids Research 17:8392; McClay et al. (2002) Analytical Biochem. 301:200-206. In certain embodiments of this technique, a mutation-specific primer is used wherein the 3′ terminal nucleotide of the primer is complementary to (i.e., capable of specifically base-pairing with) a particular variation in the target nucleic acid. If the particular variation is not present, an amplification product is not observed. Amplification Refractory Mutation System (ARMS) can also be used to detect variations (e.g., substitutions). ARMS is described, e.g., in European Patent Application Publication No. 0332435, and in Newton et al., Nucleic Acids Research, 17:7, 1989.

Other methods useful for detecting variations (e.g., substitutions) include, but are not limited to, (1) mutation-specific nucleotide incorporation assays, such as single base extension assays (see, e.g., Chen et al. (2000) Genome Res. 10:549-557; Fan et al. (2000) Genome Res. 10:853-860; Pastinen et al. (1997) Genome Res. 7:606-614; and Ye et al. (2001) Hum. Mut. 17:305-316); (2) mutation-specific primer extension assays (see, e.g., Ye et al. (2001) Hum. Mut. 17:305-316; and Shen et al. Genetic Engineering News, vol. 23, Mar. 15, 2003), including allele-specific PCR; (3) 5′ nuclease assays (see, e.g., De La Vega et al. (2002) BioTechniques 32:S48-S54 (describing the TaqMan® assay); Ranade et al. (2001) Genome Res. 11:1262-1268; and Shi (2001) Clin. Chem. 47:164-172); (4) assays employing molecular beacons (see, e.g., Tyagi et al. (1998) Nature Biotech. 16:49-53; and Mhlanga et al. (2001) Methods 25:463-71); and (5) oligonucleotide ligation assays (see, e.g., Grossman et al. (1994) Nuc. Acids Res. 22:4527-4534; patent application Publication No. US 2003/0119004 A1; PCT International Publication No. WO 01/92579 A2; and U.S. Pat. No. 6,027,889).

Variations may also be detected by mismatch detection methods. Mismatches are hybridized nucleic acid duplexes which are not 100% complementary. The lack of total complementarity may be due to deletions, insertions, inversions, or substitutions. One example of a mismatch detection method is the Mismatch Repair Detection (MRD) assay described, e.g., in Faham et al., Proc. Natl. Acad. Sci. USA 102:14717-14722 (2005) and Faham et al., Hum. Mol. Genet. 10:1657-1664 (2001). Another example of a mismatch cleavage technique is the RNase protection method, which is described in detail in Winter et al., Proc. Natl. Acad. Sci. USA, 82:7575, 1985, and Myers et al., Science 230:1242, 1985. For example, a method of the present disclosure may involve the use of a labeled riboprobe which is complementary to the human wild-type target nucleic acid. The riboprobe and target nucleic acid derived from the tissue sample are annealed (hybridized) together and subsequently digested with the enzyme RNase A which is able to detect some mismatches in a duplex RNA structure. If a mismatch is detected by RNase A, it cleaves at the site of the mismatch. Thus, when the annealed RNA preparation is separated on an electrophoretic gel matrix, if a mismatch has been detected and cleaved by RNase A, an RNA product will be seen which is smaller than the full-length duplex RNA for the riboprobe and the mRNA or DNA. The riboprobe need not be the full length of the target nucleic acid, but can a portion of the target nucleic acid, provided it encompasses the position suspected of having a variation.

In a similar manner, DNA probes can be used to detect mismatches, for example through enzymatic or chemical cleavage. See, e.g., Cotton et al., Proc. Natl. Acad. Sci. USA, 85:4397, 1988; and Shenk et al., Proc. Natl. Acad. Sci. USA, 72:989, 1975. Alternatively, mismatches can be detected by shifts in the electrophoretic mobility of mismatched duplexes relative to matched duplexes. See, e.g., Cariello, Human Genetics, 42:726, 1988. With either riboprobes or DNA probes, the target nucleic acid suspected of comprising a variation may be amplified before hybridization. Changes in target nucleic acid can also be detected using Southern hybridization, especially if the changes are gross rearrangements, such as deletions and insertions.

Restriction fragment length polymorphism (RFLP) probes for the target nucleic acid or surrounding marker genes can be used to detect variations, e.g., insertions or deletions. Insertions and deletions can also be detected by cloning, sequencing and amplification of a target nucleic acid. Single stranded conformation polymorphism (SSCP) analysis can also be used to detect base change variants of an allele. See, e.g. Orita et al., Proc. Natl. Acad. Sci. USA 86:2766-2770, 1989, and Genomics, 5:874-879, 1989. SSCP can be modified for the detection of ErbB2 somatic mutations. SSCP identifies base differences by alteration in electrophoretic migration of single stranded PCR products. Single-stranded PCR products can be generated by heating or otherwise denaturing double stranded PCR products. Single-stranded nucleic acids may refold or form secondary structures that are partially dependent on the base sequence. The different electrophoretic mobilities of single-stranded amplification products are related to base-sequence differences at SNP positions. Denaturing gradient gel electrophoresis (DGGE) differentiates SNP alleles based on the different sequence-dependent stabilities and melting properties inherent in polymorphic DNA and the corresponding differences in electrophoretic migration patterns in a denaturing gradient gel.

Somatic mutations or variations may also be detected with the use of microarrays. A microarray is a multiplex technology that typically uses an arrayed series of thousands of nucleic acid probes to hybridize with, e.g., a cDNA or cRNA sample under high-stringency conditions. Probe-target hybridization is typically detected and quantified by detection of fluorophore-, silver-, or chemiluminescence-labeled targets to determine relative abundance of nucleic acid sequences in the target. In typical microarrays, the probes are attached to a solid surface by a covalent bond to a chemical matrix (via epoxy-silane, amino-silane, lysine, polyacrylamide or others). The solid surface is for example, glass, a silicon chip, or microscopic beads. Various microarrays are commercially available, including those manufactured, for example, by Affymetrix, Inc. and Illumina, Inc.

Another method for the detection of somatic mutations is based on mass spectrometry. Mass spectrometry takes advantage of the unique mass of each of the four nucleotides of DNA. The potential mutation-containing ErbB2 nucleic acids can be unambiguously analyzed by mass spectrometry by measuring the differences in the mass of nucleic acids having a somatic mutation. MALDI-TOF (Matrix Assisted Laser Desorption Ionization-Time of Flight) mass spectrometry technology is useful for extremely precise determinations of molecular mass, such the nucleic acids containing a somatic mutation. Numerous approaches to nucleic acid analysis have been developed based on mass spectrometry. Exemplary mass spectrometry-based methods include primer extension assays, which can also be utilized in combination with other approaches, such as traditional gel-based formats and microarrays.

Sequence-specific ribozymes (U.S. Pat. No. 5,498,531) can also be used to detect somatic mutations based on the development or loss of a ribozyme cleavage site. Perfectly matched sequences can be distinguished from mismatched sequences by nuclease cleavage digestion assays or by differences in melting temperature. If the mutation affects a restriction enzyme cleavage site, the mutation can be identified by alterations in restriction enzyme digestion patterns, and the corresponding changes in nucleic acid fragment lengths determined by gel electrophoresis.

In certain embodiments of the present disclosure, protein-based detection techniques are used to detect variant proteins encoded by the genes having genetic variations as disclosed herein. Determination of the presence of the variant form of the protein can be carried out using any suitable technique known in the art, for example, electrophoresis (e.g, denaturing or non-denaturing polyacrylamide gel electrophoresis, 2-dimensional gel electrophoresis, capillary electrophoresis, and isoelectrofocusing), chromatrography (e.g., sizing chromatography, high performance liquid chromatography (HPLC), and cation-exchange HPLC), and mass spectroscopy (e.g., MALDI-TOF mass spectroscopy, electrospray ionization (ESI) mass spectroscopy, and tandem mass spectroscopy). See, e.g., Ahrer and Jungabauer (2006) J. Chromatog. B. Analyt. Technol. Biomed. Life Sci. 841: 110-122; and Wada (2002) J. Chromatog. B. 781: 291-301). Suitable techniques may be chosen based in part upon the nature of the variation to be detected. For example, variations resulting in amino acid substitutions where the substituted amino acid has a different charge than the original amino acid, can be detected by isoelectric focusing. Isoelectric focusing of the polypeptide through a gel having a pH gradient at high voltages separates proteins by their pI. The pH gradient gel can be compared to a simultaneously run gel containing the wild-type protein. In cases where the variation results in the generation of a new proteolytic cleavage site, or the abolition of an existing one, the sample may be subjected to proteolytic digestion followed by peptide mapping using an appropriate electrophoretic, chromatographic or, or mass spectroscopy technique. The presence of a variation may also be detected using protein sequencing techniques such as Edman degradation or certain forms of mass spectroscopy.

Methods known in the art using combinations of these techniques may also be used. For example, in the HPLC-microscopy tandem mass spectrometry technique, proteolytic digestion is performed on a protein, and the resulting peptide mixture is separated by reversed-phase chromatographic separation. Tandem mass spectrometry is then performed and the data collected therefrom is analyzed. (Gatlin et al. (2000) Anal. Chem., 72:757-763). In another example, nondenaturing gel electrophoresis is combined with MALDI mass spectroscopy (Mathew et al. (2011) Anal. Biochem. 416: 135-137).

In certain embodiments, the protein may be isolated from the sample using a reagent, such as antibody or peptide that specifically binds the protein, and then further analyzed to determine the presence or absence of the genetic variation using any of the techniques disclosed above.

Alternatively, the presence of the variant protein in a sample may be detected by immunoaffinity assays based on antibodies specific to proteins having genetic variations according to the present disclosure, that is, antibodies which specifically bind to the protein having the variation, but not to a form of the protein which lacks the variation. Such antibodies can be produced by any suitable technique known in the art. Antibodies can be used to immunoprecipitate specific proteins from solution samples or to immunoblot proteins separated by, e.g., polyacrylamide gels. Immunocytochemical methods can also be used in detecting specific protein variants in tissues or cells. Other well known antibody-based techniques can also be used including, e.g., enzyme-linked immunosorbent assay (ELISA), radioimmuno-assay (RIA), immunoradiometric assays (IRMA) and immunoenzymatic assays (IEMA), including sandwich assays using monoclonal or polyclonal antibodies. See e.g., U.S. Pat. Nos. 4,376,110 and 4,486,530.

Diagnosis and Prognosis of Cancer

The present disclosure provides methods for the diagnosis or prognosis of cancer in a subject by detecting the presence in a sample from the subject of one or more somatic mutations or variations associated with cancer as disclosed herein. Somatic mutations or variations for use in the methods of the present disclosure include variations in ErbB2, or the genes encoding this protein. In certain embodiments, the somatic mutation is in genomic DNA that encodes a gene (or its regulatory region). In certain embodiments, the somatic mutation is a substitution, an insertion, or a deletion in the gene coding for ErbB2. In an embodiment, the variation is a mutation that results in an amino acid substitution at one or more of the positions identified in Table 1 in the amino acid sequence of ErbB2 (SEQ ID NO:2). In certain embodiments, the variation is a mutation that results in an amino acid substitution at one or more of V659, R667, R678, G660 and Q709 in the amino acid sequence of ErbB2 (SEQ ID NO:2). In certain embodiments, the substitution is at least one of V659E, R667Q, R678Q, G660D, G660R and Q709L in the amino acid sequence of ErbB2 (SEQ ID NO:2). In certain embodiments, the mutation indicates the presence of an ErbB2-positive cancer selected from the group consisting of gastric, colon, esophageal, rectal, cecum, colorectal, non-small-cell lung (NSCLC) adenocarcinoma, NSCLC (Squamous carcinoma), renal carcinoma, melanoma, ovarian, lung large cell, small-cell lung cancer (SCLC), hepatocellular (HCC), lung cancer, and pancreatic cancer.

In certain embodiments, the variation is a mutation that results in an amino acid substitution at one or more of V659E, R667Q, R678Q, G660D, G660R in the amino acid sequence of ErbB2 (SEQ ID NO:2). For example, but not by way of limitation, the substitution is at least one of V659E, R667Q, R678Q, G660D, G660R and Q709L in the amino acid sequence of ErbB2 (SEQ ID NO:2). In certain embodiments, the ErbB2 mutation indicates the presence of gastrointestinal cancer, e.g., gastric, colon, esophageal, rectal, cecum, and colorectal cancer.

In certain embodiments, the ErbB2 substitution is at V659. For example, but not by way of limitation, the substitution is V659E. In certain embodiments, the mutation indicates the presence of colon cancer.

In certain embodiments, the ErbB2 substitution is at V659. In certain embodiments, the substitution is V659E. For example, but not by way of limitation, the mutation indicates the presence of breast cancer.

In certain embodiments, the ErbB2 substitution is at R667. In certain embodiments, the substitution is R667Q. For example, but not by way of limitation, the mutation indicates the presence of gastric cancer or colon cancer.

In certain embodiments, the ErbB2 substitution is at R667. In certain embodiments, the substitution is R667Q. For example, but not by way of limitation, the mutation indicates the presence of breast cancer.

In certain embodiments, the ErbB2 substitution is at R678. In certain embodiments, the substitution is R678Q. For example, but not by way of limitation, the mutation indicates the presence of gastric cancer.

In certain embodiments, the ErbB2 substitution is at R678. In certain embodiments, the substitution is R678Q. For example, but not by way of limitation, the mutation indicates the presence of breast cancer.

In certain embodiments, the ErbB2 substitution is at G660. In certain embodiments, the substitution is G660D or G660R. For example, but not by way of limitation, the mutation indicates the presence of gastric cancer.

In certain embodiments, the ErbB2 substitution is at G660. For example, but not by way of limitation, the substitution is G660D or G660R. For example, but not by way of limitation, the mutation indicates the presence of breast cancer.

In certain embodiments, the ErbB2 substitution is at Q709. For example, but not by way of limitation, the substitution is Q709L. In certain embodiments, the mutation indicates the presence of colon cancer.

In certain embodiments, the ErbB2 substitution is at Q709. For example, but not by way of limitation, the substitution is Q709L. In certain embodiments, the mutation indicates the presence of breast cancer.

In certain embodiments, the ErbB2 substitution is at V659. For example, but not by way of limitation, the substitution is V659E. In certain embodiments, the mutation indicates the presence of lung cancer (non-small-cell lung (NSCLC) adenocarcinoma) or lung cancer (non-small-cell lung (NSCLC) squamous carcinoma).

In certain embodiments, the ErbB2 substitution is at R667. For example, but not by way of limitation, the substitution is R667Q. In certain embodiments, the mutation indicates the presence of lung cancer (non-small-cell lung (NSCLC) adenocarcinoma) or lung cancer (non-small-cell lung (NSCLC) squamous carcinoma).

In certain embodiments, the ErbB2 substitution is at R678. For example, but not by way of limitation, the substitution is R678Q. In certain embodiments, the mutation indicates the presence of lung cancer (non-small-cell lung (NSCLC) adenocarcinoma) or lung cancer (non-small-cell lung (NSCLC) squamous carcinoma).

In certain embodiments, the ErbB2 substitution is at G660. For example, but not by way of limitation, the substitution is G660D or G660R. In certain embodiments, the mutation indicates the presence of lung cancer (non-small-cell lung (NSCLC) adenocarcinoma) or lung cancer (non-small-cell lung (NSCLC) squamous carcinoma).

In certain embodiments, the ErbB2 substitution is at Q709. For example, but not by way of limitation, the substitution is Q709L. In certain embodiments, the mutation indicates the presence of lung cancer (non-small-cell lung (NSCLC) adenocarcinoma) or lung cancer (non-small-cell lung (NSCLC) squamous carcinoma).

In certain embodiments, the at least one variation is an amino acid substitution, insertion, truncation, or deletion in ErbB2. In certain embodiments, the variation is an amino acid substitution. Any one or more of these variations may be used in any of the methods of detection, diagnosis and prognosis described below.

In certain embodiments, the present disclosure provides a method for detecting the presence or absence of a somatic mutation indicative of cancer in a subject, comprising: (a) contacting a sample from the subject with a reagent capable of detecting the presence or absence of a somatic mutation in an ErbB2 gene; and (b) determining the presence or absence of the mutation, wherein the presence of the mutation indicates that the subject is afflicted with, or at risk of developing, cancer.

The reagent for use in the method may be an oligonucleotide, a DNA probe, an RNA probe, and a ribozyme. In certain embodiments, the reagent is labeled. Labels may include, for example, radioisotope labels, fluorescent labels, bioluminescent labels or enzymatic labels. Radionuclides that can serve as detectable labels include, for example, 1-131, 1-123, 1-125, Y-90, Re-188, Re-186, At-211, Cu-67, Bi-212, and Pd-109.

The present disclosure provides a method for detecting a somatic mutation indicative of cancer in a subject. In certain embodiments, the method for detecting a somatic mutation indicative of cancer in a subject comprises determining the presence or absence of a somatic mutation in an ErbB2 gene in a biological sample from a subject, wherein the presence of the mutation indicates that the subject is afflicted with, or at risk of developing, cancer. In certain embodiments of the method, detection of the presence of the one or more somatic mutations is carried out by a process selected from the group consisting of direct sequencing, mutation-specific probe hybridization, mutation-specific primer extension, mutation-specific amplification, mutation-specific nucleotide incorporation, 5′ nuclease digestion, molecular beacon assay, oligonucleotide ligation assay, size analysis, and single-stranded conformation polymorphism. In certain embodiments, nucleic acids from the sample are amplified prior to determining the presence of the one or more mutations.

The present disclosure further provides a method for diagnosing or prognosing cancer in a subject. In certain embodiments, the method comprises (a) contacting a sample from the subject with a reagent capable of detecting the presence or absence of a somatic mutation in an ErbB2 gene; and (b) determining the presence or absence of the mutation, wherein the presence of the mutation indicates that the subject is afflicted with, or at risk of developing, cancer. In certain embodiments, the methods include determining the presence or absence of a somatic mutation in an ErbB2 gene in a biological sample from a subject, wherein the presence of the genetic variation indicates that the subject is afflicted with, or at risk of developing, cancer.

In certain embodiments, the method of diagnosing or prognosing cancer in a subject, can include (a) obtaining a nucleic-acid containing sample from the subject, and (b) analyzing the sample to detect the presence of at least one somatic mutation in an ErbB2 gene, wherein the presence of the genetic variation indicates that the subject is afflicted with, or at risk of developing, cancer.

In certain embodiments, the method of diagnosis or prognosis further comprises subjecting the subject to one or more additional diagnostic tests for cancer, for example, screening for one or more additional markers, or subjecting the subject to imaging procedures.

In certain embodiments, the above methods further comprise detecting in the sample the presence of at least one somatic mutation. In certain embodiments, the presence of a first somatic mutation together with the presence of at least one additional somatic mutation is indicative of an increased risk of cancer compared to a subject having the first somatic mutation and lacking the presence of the at least one additional somatic mutation.

The present disclosure further provides methods for identifying a subject having an increased risk of the diagnosis of cancer. In certain embodiments, the methods includes (a) determining the presence or absence of a first somatic mutation in an ErbB2 gene in a biological sample from a subject; and (b) determining the presence or absence of at least one additional somatic mutation, wherein the presence of the first and at least one additional somatic mutations indicates that the subject has an increased risk of the diagnosis of cancer as compared to a subject lacking the presence of the first and at least one additional somatic mutation.

Also provided is a method of aiding diagnosis and/or prognosis of a sub-phenotype of cancer in a subject, the method comprising detecting in a biological sample derived from the subject the presence of a somatic mutation in a gene encoding ErbB2.

The present disclosure further provides a method of predicting the response of a subject to a cancer therapeutic agent that targets an ErbB receptor, comprising detecting in a biological sample obtained from the subject a somatic mutation that results in an amino acid variation in the amino acid sequence of ErbB2 (SEQ ID NO: 2), wherein the presence of the somatic mutation is indicative of a response to a therapeutic agent that targets an ErbB receptor. In certain embodiments, the therapeutic agent is an ErbB antagonist or binding agent, for example, an anti-ErbB antibody.

A biological sample for use in any of the methods described above may be obtained using certain methods known to those skilled in the art. Biological samples may be obtained from vertebrate animals, and in particular, mammals. In certain embodiments, a biological sample comprises a cell or tissue. Variations in target nucleic acids (or encoded polypeptides) may be detected from a tissue sample or from other body samples such as blood, serum, urine, sputum, saliva, mucosa, and tissue. By screening such body samples, a simple early diagnosis can be achieved for diseases such as cancer. In addition, the progress of therapy can be monitored more easily by testing such body samples for variations in target nucleic acids (or encoded polypeptides). In certain embodiments, the biological sample is obtained from an individual suspected of having cancer.

Subsequent to the determination that a subject, or biological sample obtained from the subject, comprises a somatic mutation disclosed herein, it is contemplated that an effective amount of an appropriate cancer therapeutic agent may be administered to the subject to treat cancer in the subject.

Also provided are methods for aiding in the diagnosis of cancer in a mammal by detecting the presence of one or more variations in nucleic acid comprising a somatic mutation in ErbB2, according to the method described above.

In certain embodiments, a method is provided for predicting whether a subject with cancer will respond to a therapeutic agent by determining whether the subject comprises a somatic mutation in ErbB2, according to the method described above.

Also provided are methods for assessing predisposition of a subject to develop cancer by detecting presence or absence in the subject of a somatic mutation in ErbB2.

Also provided are methods of sub-classifying cancer in a mammal, the method comprising detecting the presence of a somatic mutation in ErbB2.

Also provided are methods of identifying a therapeutic agent effective to treat cancer in a patient subpopulation, the method comprising correlating efficacy of the agent with the presence of a somatic mutation in ErbB2.

Additional methods provide information useful for determining appropriate clinical intervention steps, if and as appropriate. Therefore, in certain embodiments of a method of the present disclosure, the method further comprises a clinical intervention step based on results of the assessment of the presence or absence of an ErbB2 somatic mutation associated with cancer as disclosed herein. For example, appropriate intervention may involve prophylactic and treatment steps, or adjustment(s) of any then-current prophylactic or treatment steps based on genetic information obtained by a method of the present disclosure.

As would be evident to one skilled in the art, in any method described herein, while detection of presence of a somatic mutation would positively indicate a characteristic of a disease (e.g., presence or subtype of a disease), non-detection of a somatic mutation would also be informative by providing the reciprocal characterization of the disease.

### Treatment of Cancer

The present disclosure provides methods of treating a patient who has an ErbB2-positive cancer, wherein the cancer comprises a mutation in the JM or TM domains of the ErbB2 receptor. In certain embodiments, the ErbB2-positive cancer comprises at least one mutation shown in Table 1. In certain embodiments, the method of treating cancer in a patient comprises the steps of obtaining a biological sample from the patient, examining the biological sample for the presence or absence of an ErbB2 somatic mutation as disclosed herein, and upon determining the presence or absence of the mutation in said tissue or cell sample, administering an effective amount of an appropriate therapeutic agent to said patient. Optionally, the methods comprise administering an effective amount of a targeted cancer therapeutic agent to said mammal. For example, but not by way of limitation, if an ErbB2 somatic mutation is detected in the biological sample, the method can include the administration of an effective amount of a Her inhibitor.

In certain embodiments, a method for the treatment of a subject having cancer can include obtaining a sample of the cancer from the subject and detecting the presence of an ErbB2 somatic mutation in the sample, wherein if an ErbB2 somatic mutation is detected, then administering a Her inhibitor to the subject. In certain embodiments, the ErbB2 mutation includes a mutation in the TM region and/or JM region of the ErbB2 receptor. In certain embodiments, the ErbB2 mutation is a mutation of at least one of amino acids V659, G660 R667, R678, Q709 or a combination thereof. For example, but not by way of limitation, the ErbB2 mutation is selected from the group consisting of V659E, G660D, G660R, R667Q, R678Q, Q709L and a combination thereof.

Also provided are methods of treating cancer in a subject in whom an ErbB2 somatic mutation is known to be present, the method comprising administering to the subject a therapeutic agent effective to treat cancer. In certain embodiments, the ErbB2 mutation is one provided in Table 1. In certain embodiments, the ErbB2 mutation is a mutation of at least one of amino acids V659, G660 R667, R678, Q709 or a combination thereof. For example, but not by way of limitation, the ErbB2 mutation is selected from the group consisting of V659E, G660D, G660R, R667Q, R678Q, Q709L and a combination thereof. Also provided are methods of treating a cancer subject who is of a specific cancer patient subpopulation comprising administering to the subject an effective amount of a therapeutic agent that is approved as a therapeutic agent for said subpopulation, wherein the subpopulation is characterized at least in part by association with an ErbB2 somatic mutation. In certain embodiments, the ErbB2 mutation is one provided in Table 1. In certain embodiments, the ErbB2 mutation is a mutation of at least one of amino acids V659, G660 R667, R678, Q709 or a combination thereof. For example, but not by way of limitation, the ErbB2 mutation is selected from the group consisting of V659E, G660D, G660R, R667Q, R678Q, Q709L and a combination thereof.

Also provided are methods for selecting a patient suffering from cancer for treatment with a cancer therapeutic agent comprising detecting the presence of an ErbB2 somatic mutation. In certain embodiments, patients are selected for treatment with Herceptin or Pertuzamab based on the presence of one or more of the mutations disclosed in Table 1.

In certain embodiments, a patient suffering from a cancer which comprises a mutation in the TM region of the ErbB2 receptor is selected for treatment with trastuzumab or trastuzumab-MCC-DM1 (T-DM1). In certain embodiments, a patient suffering from a cancer which comprises a mutation at at least one of amino acids V659 or G660 of the TM domain of the ErbB2 receptor is selected for treatment with trastuzumab or trastuzumab-MCC-DM1 (T-DM1). In certain embodiments, the cancer comprises the mutation V659E. In certain embodiments, the cancer comprises the mutation G660D. In certain embodiments, the cancer comprises the mutation G660R. In certain embodiments, the patient suffering from a cancer which comprises a mutation in the TM region of the ErbB2 receptor is administered an effective amount of trastuzumab. In certain embodiments, the patient suffering from a cancer which comprises a mutation in the TM region of the ErbB2 receptor is administered an effective amount of trastuzumab-MCC-DM1 (T-DM1).

In certain embodiments, a patient suffering from a cancer which comprises a mutation in the JM region of the ErbB2 receptor is selected for treatment with trastuzumab, trastuzumab-MCC-DM1 (T-DM1), or pertuzumab. In certain embodiments, a patient suffering from a cancer which comprises a mutation at least one of amino acids R667, R678 or Q709 of the JM domain of the ErbB2 receptor is selected for treatment with trastuzumab, trastuzumab-MCC-DM1 (T-DM1), or pertuzumab. In certain embodiments, the cancer comprises the mutation R667Q. In certain embodiments, the cancer comprises the mutation R678Q. In yet certain embodiments, the cancer comprises the mutation Q709L. In certain embodiments, the patient suffering from a cancer which comprises a mutation in the JM region of the ErbB2 receptor patient is administered an effective amount of trastuzumab. In certain embodiments, the patient suffering from a cancer which comprises a mutation in the JM region of the ErbB2 receptor is administered an effective amount of trastuzumab-MCC-DM1 (T-DM1). In certain embodiments, the patient suffering from a cancer which comprises a mutation in the JM region of the ErbB2 receptor is administered an effective amount of pertuzumab.

The present disclosure provides methods of treating an individual having an Her2/ErbB2 cancer identified by one or more of the somatic mutations described herein. In certain embodiments, the method comprises the step of administering to the individual an effective amount of a Her inhibitor. In certain embodiments, the Her inhibitor is an antibody which binds to a Her receptor. In certain embodiment, the antibody binds to an ErbB2 receptor. In certain embodiments, the cancer treated by the Her inhibitor is gastric, colon, esophageal, rectal, cecum, colorectal, non-small-cell lung (NSCLC) adenocarcinoma, NSCLC (Squamous carcinoma), renal carcinoma, melanoma, ovarian, lung large cell, small-cell lung cancer (SCLC), hepatocellular (HCC), lung cancer, and pancreatic cancer.

In another aspect, the present disclosure provides an anti-cancer therapeutic agent for use in a method of treating an ErbB2-positive cancer in a subject, said method comprising (i) detecting in a biological sample obtained from the subject the presence or absence of an amino acid mutation in a nucleic acid sequence encoding ErbB2, wherein the mutation results in an amino acid change at at least one position of the ErbB2 amino acid sequence (as described herein), wherein the presence of the mutation is indicative of the presence of cancer in the subject from which the sample was obtained; and (ii) if a mutation is detected in the nucleic acid sequence, administering to the subject an effective amount of the anti-cancer therapeutic agent.

Another aspect of the present disclosure provides for a method of inhibiting a biological activity of a Her receptor in an individual comprising administering to the individual an effective amount of a Her inhibitor. In certain embodiments, the Her receptor is a Her2 receptor expressed by cancer cells in the individual. In certain embodiments, the Her inhibitor is a Her antibody comprising an antigen-binding domain that specifically binds to at least Her2.

In certain embodiments, the present disclosure provides a method for lengthening the period of survival of a subject having a cancer that includes an ErbB2 somatic mutation. In certain embodiments, the method includes administering, to the subject, a therapeutically effective amount of a HER inhibitor, disclosed herein. In certain embodiments, the period of survival of a subject having cancer can be lengthened by about 1 week, about 2 weeks, about 3 weeks, about 1 month, about 2 months, about 4 months, about 6 months, about 8 months, about 10 months, about 12 months, about 14 months, about 18 months, about 20 months, about 2 years, about 3 years, about 5 years or more using the disclosed methods.

In another aspect, the present disclosure provides several different types of suitable Her inhibitors for use in the methods of treatment. In certain embodiments, the Her inhibitor is selected from the group consisting of trastuzumab (an anti-ErbB2 antibody that binds ErbB2 domain IV), Trastuzumab-MCC-DM1 (T-DM1), pertuzumab (an anti-ErbB2 antibody that binds ErbB2 domain II and prevents dimerization) and a combination thereof. Additional non-limiting examples of Her inhibitors include lapatinib, afatinib and neratinib.

The present disclosure further provides for a Her antibody for use as a medicament. Another aspect of the present disclosure provides for a Her antibody for use in the manufacture of a medicament. The medicament can be used, in certain embodiments, to treat an ErbB2/Her2 cancer identified by one or more of the somatic mutations described herein. In certain embodiments, the Her antibody comprises an antigen-binding domain that specifically binds to Her2, or to Her2 and at least one additional Her receptor.

Trastuzumab (CAS 180288-69-1, HERCEPTIN®, huMAb4 D5-8, rhuMAb Her2, Genentech) is a recombinant DNA-derived, IgG1 kappa, monoclonal antibody that is a humanized version of a murine anti-Her2 antibody (4 D5) that selectively binds with high affinity in a cell-based assay (Kd=5 nM) to the extracellular domain of Her2 (U.S. Pat. Nos. 5,677,171; 5,821,337; 6,054,297; 6,165,464; 6,339,142; 6,407,213; 6,639,055; 6,719,971; 6,800,738; 7,074,404; Coussens et al (1985) Science 230:1132-9; Slamon et al (1989) Science 244:707-12; Slamon et al (2001) New Engl. J. Med. 344:783-792). Trastuzumab has been shown, in both in vitro assays and in animals, to inhibit the proliferation of human tumor cells that overexpress Her2 (Hudziak et al (1989) Mol Cell Biol 9:1165-72; Lewis et al (1993) Cancer Immunol Immunother; 37:255-63; Baselga et al (1998) Cancer Res. 58:2825-2831). Trastuzumab is a mediator of antibody-dependent cellular cytotoxicity, ADCC (Lewis et al (1993) Cancer Immunol Immunother 37(4):255-263; Hotaling et al (1996) [abstract]. Proc. Annual Meeting Am Assoc Cancer Res; 37:471; Pegram M D, et al (1997) [abstract]. Proc Am Assoc Cancer Res; 38:602; Sliwkowski et al (1999) Seminars in Oncology 26(4), Suppl 12:60-70; Yarden Y. and Sliwkowski, M. (2001) Nature Reviews: Molecular Cell Biology, Macmillan Magazines, Ltd., Vol. 2:127-137).

HERCEPTIN®. was approved in 1998 for the treatment of patients with Her2-overexpressing metastatic breast cancers (Baselga et al, (1996) J. Clin. Oncol. 14:737-744) that have received extensive prior anti-cancer therapy, and has since been used in over 300,000 patients (Slamon D J, et al. N Engl J Med 2001; 344:783-92; Vogel C L, et al. J Clin Oncol 2002; 20:719-26; Marty M, et al. J Clin Oncol 2005; 23:4265-74; Romond E H, et al. T N Engl J Med 2005; 353:1673-84; Piccart-Gebhart M J, et al. N Engl J Med 2005; 353:1659-72; Slamon D, et al. [abstract]. Breast Cancer Res Treat 2006, 100 (Suppl 1): 52). In 2006, the FDA approved HERCEPTIN®. (trastuzumab, Genentech Inc.) as part of a treatment regimen containing doxorubicin, cyclophosphamide and paclitaxel for the adjuvant treatment of patients with Her2-positive, node-positive breast cancer.

Trastuzumab-MCC-DM1 (T-DM1, trastuzumab emtansine, ado-trastuzumab emtansine, KADCYLA®), a novel antibody-drug conjugate (ADC) for the treatment of Her2-positive breast cancer, is composed of the cytotoxic agent DM1 (a thiol-containing maytansinoid anti-microtubule agent) conjugated to trastuzumab at lysine side chains via an MCC linker, with an average drug load (drug to antibody ratio) of about 3.5. After binding to Her2 expressed on tumor cells, T-DM1 undergoes receptor-mediated internalization, resulting in intracellular release of cytotoxic catabolites containing DM1 and subsequent cell death.

The U.S. Food and Drug Administration approved ado-trastuzumab emtansine, marketed under the tradename KADCYLA®, on Feb. 22, 2013 for the treatment of patients with Her2-positive, metastatic breast cancer who previously received treatment with trastuzumab and a taxane.

Pertuzumab (also known as recombinant humanized monoclonal antibody 2C4, rhuMAb 2C4, PERJETA®, Genentech, Inc, South San Francisco) represents the first in a new class of agents known as Her dimerization inhibitors (HDI) and functions to inhibit the ability of Her2 to form active heterodimers or homodimers with other Her receptors (such as EGFR/Her1, Her2, Her3 and Her4). See, for example, Harari and Yarden Oncogene 19:6102-14 (2000); Yarden and Sliwkowski. Nat Rev Mol Cell Biol 2:127-37 (2001); Sliwkowski Nat Struct Biol 10:158-9 (2003); Cho et al. Nature 421:756-60 (2003); and Malik et al. Pro Am Soc Cancer Res 44:176-7 (2003)

Pertuzumab blockade of the formation of Her2-Her 3 heterodimers in tumor cells has been demonstrated to inhibit critical cell signaling, which results in reduced tumor proliferation and survival (Agus et al. Cancer Cell 2:127-37 (2002)).

Pertuzumab has been evaluated in Phase II studies in combination with trastuzumab in patients with Her2-positive metastatic breast cancer who have previously received trastuzumab for metastatic disease. One study, conducted by the National Cancer Institute (NCO, enrolled 11 patients with previously treated Her2-positive metastatic breast cancer. Two out of the 11 patients exhibited a partial response (PR) (Baselga et al., J Clin Oncol 2007 ASCO Annual Meeting Proceedings; 25:18 S (June 20 Supplement): 1004. The results of a Phase II neoadjuvant study evaluating the effect of a novel combination regimen of pertuzumab and trastuzumab plus chemotherapy (Docetaxel) in women with early-stage Her2-positive breast cancer, presented at the CTRC-AACR San Antonio Breast Cancer Symposium (SABCS), Dec. 8-12, 2010, showed that the two Her2 antibodies plus Docetaxel given in the neoadjuvant setting prior to surgery significantly improved the rate of complete tumor disappearance (pathological complete response rate, pCR, of 45.8 percent) in the breast by more than half compared to trastuzumab plus Docetaxel (pCR of 29. 0 percent), p=0.014.

Pertuzumab, marketed under the tradename PERJETA®, was approved in 2012 for the treatment of patients with advanced or late-stage (metastatic) Her2-positive breast cancer. Her2-positive breast cancers have increased amounts of the Her2 protein that contributes to cancer cell growth and survival.

A therapeutic agent for the treatment of cancer may be incorporated into compositions, which in certain embodiments are suitable for pharmaceutical use. Such compositions typically comprise the peptide or polypeptide, and an acceptable carrier, for example one that is pharmaceutically acceptable. A “pharmaceutically acceptable carrier” includes any and all solvents, dispersion media, coatings, antibacterial and antifungal agents, isotonic and absorption delaying agents, and the like, compatible with pharmaceutical administration (Gennaro, Remington: The science and practice of pharmacy. Lippincott, Williams & Wilkins, Philadelphia, Pa. (2000)). Examples of such carriers or diluents include, but are not limited to, water, saline, Finger's solutions, dextrose solution, and 5% human serum albumin. Liposomes and non-aqueous vehicles such as fixed oils may also be used. Except when a conventional media or agent is incompatible with an active compound, use of these compositions is contemplated. Supplementary active compounds can also be incorporated into the compositions.

A therapeutic agent of the present disclosure (and any additional therapeutic agent for the treatment of cancer) can be administered by any suitable means, including parenteral, intrapulmonary, intrathecal and intranasal, and, if desired for local treatment, intralesional administration. Parenteral infusions include, e.g., intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Effective dosages and schedules for administering cancer therapeutic agents may be determined empirically, and making such determinations is within the skill in the art. Single or multiple dosages may be employed. When in vivo administration of a cancer therapeutic agent is employed, normal dosage amounts may vary from about 10 ng/kg to up to 100 mg/kg of mammal body weight or more per day, preferably about 1 μg/kg/day to 10 mg/kg/day, depending upon the route of administration. Guidance as to particular dosages and methods of delivery is provided in the literature; see, for example, U.S. Pat. Nos. 4,657,760; 5,206,344; or 5,225,212.

### Combination Therapy

It is contemplated that combination therapies may be employed in the methods. The combination therapy may include but are not limited to, administration of two or more cancer therapeutic agents. Administration of the therapeutic agents in combination typically is carried out over a defined time period (usually minutes, hours, days or weeks depending upon the combination selected). Combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner.

The therapeutic agent can be administered by the same route or by different routes. For example, an ErbB antagonist in the combination may be administered by intravenous injection while a chemotherapeutic agent in the combination may be administered orally. Alternatively, for example, both of the therapeutic agents may be administered orally, or both therapeutic agents may be administered by intravenous injection, depending on the specific therapeutic agents. The sequence in which the therapeutic agents are administered also varies depending on the specific agents.

In certain embodiments, the present disclosure provides a method of treating an individual having an ErbB2/Her2 cancer identified by one or more of the somatic mutations described herein, wherein the method of treatment comprises administering more than one ErbB inhibitor. In certain embodiments, the method comprises administering more than one ErbB2 inhibitor. For example, but not by way of limitation, the methods of treatment disclosed herein can include the administration of a combination of trastuzumab, Trastuzumab-MCC-DM1 (T-DM1), pertuzumab, lapatinib, afatinib, neratinib. In certain embodiments, the methods of treatment can include the administration of trastuzumab or Trastuzumab-MCC-DM1 (T-DM1) and pertuzumab. In certain embodiments, the methods of treatment disclosed herein can include the administration of trastuzumab and pertuzumab. Alternatively, the methods of treatment disclosed herein can include the administration of Trastuzumab-MCC-DM1 (T-DM1) and pertuzumab. In certain embodiments, the methods of treatment can include the administration of trastuzumab or Trastuzumab-MCC-DM1 (T-DM1) and lapatinib, afatinib or neratinib.

### Kits

For use in the applications described or suggested herein, kits or articles of manufacture are also provided. Such kits may comprise a carrier means being compartmentalized to receive in close confinement one or more container means such as vials, tubes, and the like, each of the container means comprising one of the separate elements to be used in the method. For example, one of the container means may comprise a probe that is or can be detectably labeled. Such probe may be a polynucleotide specific for a polynucleotide comprising an ErbB2 somatic mutation associated with cancer as disclosed herein. Where the kit utilizes nucleic acid hybridization to detect a target nucleic acid, the kit may also have containers containing nucleotide(s) for amplification of the target nucleic acid sequence and/or a container comprising a reporter means, such as a biotin-binding protein, such as avidin or streptavidin, bound to a reporter molecule, such as an enzymatic, florescent, or radioisotope label. In certain embodiments, the kits of the present disclosure comprise one or more ErbB2-positive cancer detecting agents as described herein. In certain embodiments, the kit further comprises a therapeutic agent (e.g., an ErbB2 inhibitor), as described herein.

In certain embodiments, the kit may comprise a labeled agent capable of detecting a polypeptide comprising an ErbB2 somatic mutation associated with cancer as disclosed herein. Such agents may be antibodies which bind the polypeptide. Such agents may be peptides which binds the polypeptide. The kit may comprise, for example, a first antibody (e.g., attached to a solid support) which binds to a polypeptide comprising a genetic variant as disclosed herein; and, optionally, a second, different antibody which binds to either the polypeptide or the first antibody and is conjugated to a detectable label.

In certain embodiments, kits of the present disclosure can include the container described above and one or more other containers comprising materials desirable from a commercial and user standpoint, including buffers, diluents, filters, needles, syringes, and package inserts with instructions for use. A label may be present on the container to indicate that the composition is used for a specific therapy or non-therapeutic application, and may also indicate directions for either in vivo or in vitro use, such as those described above. Other optional components in the kit include one or more buffers (e.g., block buffer, wash buffer, substrate buffer, etc), other reagents such as substrate (e.g., chromogen) which is chemically altered by an enzymatic label, epitope retrieval solution, control samples (positive and/or negative controls), control slide(s) etc.

In another aspect, the present disclosure provides the use of an ErbB2-positive cancer detecting agent in the manufacture of a kit for detecting cancer in a subject. In certain embodiments, the detection of an ErbB2-positive cancer comprises detecting in a biological sample obtained from the subject the presence or absence of an amino acid mutation in a nucleic acid sequence encoding ErbB2, wherein the mutation results in an amino acid change at at least one position of the ErbB2 amino acid sequence (as described herein), wherein the presence of the mutation is indicative of the presence of cancer in the subject from which the sample was obtained. In certain embodiments, the ErbB2-positive cancer detecting agent specifically detects an ErbB2 nucleic acid transcript or protein that encodes or includes one or more mutations presented in Table 1 and does not detect a wildtype ErbB2 nucleic acid transcript or protein.

### Methods of Marketing

The disclosure herein also encompasses a method for marketing the disclosed methods of diagnosis or prognosis of cancer comprising advertising to, instructing, and/or specifying to a target audience, the use of the disclosed methods.

Marketing is generally paid communication through a non-personal medium in which the sponsor is identified and the message is controlled. Marketing for purposes herein includes publicity, public relations, product placement, sponsorship, underwriting, and the like. This term also includes sponsored informational public notices appearing in any of the print communications media.

The marketing of the diagnostic method herein may be accomplished by any means. Examples of marketing media used to deliver these messages include television, radio, movies, magazines, newspapers, the internet, and billboards, including commercials, which are messages appearing in the broadcast media.

The type of marketing used will depend on many factors, for example, on the nature of the target audience to be reached, e.g., hospitals, insurance companies, clinics, doctors, nurses, and patients, as well as cost considerations and the relevant jurisdictional laws and regulations governing marketing of medicaments and diagnostics. The marketing may be individualized or customized based on user characterizations defined by service interaction and/or other data such as user demographics and geographical location.

The following examples are offered for illustrative purposes only, and are not intended to limit the scope of the present disclosure in any way.

All patent and literature references cited in the present specification are hereby incorporated by reference in their entirety.

### EXAMPLES

### Example—Oncogenic ErbB2 Mutations in Tumorigenesis

Given the importance of ErbB2 in human cancers, we systematically surveyed human cancers and identified recurring somatic mutations in the transmembrane (TM) and juxtamembrane (JM) domains of ErbB2, as well as the regions adjacent to the JM/TM domains, and also show that these mutations are transforming. Further, we evaluated targeted therapeutics in ErbB2-mutant driven cell-based and animal models of cancer and show them to be effective in blocking ErbB2-mutant driven oncogenesis.

### Materials and Methods

Tumor DNA, Mutation Identification

Tumor-DNA mutations were identified based on observed mutation frequency from ErbB2 mutations in tumors in patients and/or those in the proximity of the observed mutations in the TM/JM domain region and adjoining segments as indicated (Table 1).

Cell Lines

The IL-3-dependent mouse pro-B cell line BaF3 was purchased from ATCC (American Type Culture Collection, Manassas, Va.). BaF3 cells were maintained in RPMI 1640 supplemented with 10% (v/v) fetal bovine serum (Thermo Fisher Scientific, IL), 2 mM L-glutamine, 100 U/ml penicillin, 100 mg/ml streptomycin (complete RPMI) and 2 ng/mL mouse IL-3.

Retrovirus Preparation and Generation of Stable Cell Lines

The pLPCX retroviral vector (Clontech, CA) expressing full length Her2 wild type (WT) with an N-terminal herpes simplex glycoprotein D (gD) tag was used for site directed mutagenesis. Her2 mutants were generated using Quikchange site-directed mutagenesis Kit (Agilent, CA; Table 1). The retrovirus generated using wildtype (WT) or mutant Her2 plasmids as described previously (Jaiswal et al., 2009) was used to generate stable BaF3 cell lines. Stable cells were selected using Phoenix cells were plated in 6 well plate a day prior to infection. BaF3 cells were cultured in complete RPMI media supplemented with recombinant murine IL-3 (mIL-3) and puromycin (1 μg/ml).

Cell Survival Assay and Western Blots

BaF3 cells survival assay was performed as described previously (Jaiswal et al., 2011). Briefly, stable cells expressing Her2 WT or mutants were washed twice times with 1× PBS and plated in 96-well plates (10,000 cells/well) in replicates of 12 in complete RPMI medium without IL-3. Cell viability was measured using the Cell Titer Glo Luminescence Cell Viability Kit (Promega, CA), and plates were read on a Synergy 2 (Biotek Instruments) luminescence plate reader. Relative survival reported was calculated as a ratio of relative luciferase activity (RLU) at day 4 over RLU measured at the day 0. The mutant Her2 constructs were tested in BaF3 either alone or in the presence of WT Flag tagged Her2 as indicated.

Expression of tagged Her2 was tested using western blot as previously described (Jaiswal et al., 2011)

### ErbB2 Inhibitors Testing

BaF3 cells stably expressing ErbB2 G660D, G660R, V659E, R678Q, or Q709L mutants were washed twice with PBS and suspended in RPMI lacking IL-3. About 10000 cells were plated in each well of 96 well plates in 100 ul of IL-3-free RPMI medium and treated with either a Her2 antibody (Trastuzumab or Pertuzumab) or an ErbB2 kinase inhibitory small molecule drug (lapatinib, afatinib or neratinib) as indicated. Viable cell number was assessed 4 days after treatment using Cell Titer-Glo Luminescent cell viability assay kit (Promega, WI). Non-linear regression plot of antibodies and their fractions or inhibitors were generated and calculation of IC50 was performed using GraphPad Prism 5.00 (GraphPad Software, CA). Data are presented as mean±SEM of at least 3-4 replicate of a representative experiment that was repeated at least thrice.

Animal Studies

BaF3 cells (2×10⁶) expressing the ErbB2 wild-type or mutants may also be implanted into 8-12 week old Balb/C nude mice by tail vein injection. For in vivo antibody efficacy study, mice can be treated with 40 mg/kg QW anti-Ragweed (control), 10 mg/kg QW trastuzumab, or 10 mg/kg QW pertuzumab starting on day 4 after cell implant. A majority of the mice can be followed for survival and some can be used for necropsy at day 20 to assess disease progression by histological analysis of bone marrow, spleen and liver. Bone marrow and spleen single cell suspension obtained from these animals may also analyzed for the presence and proportion of GFP positive BaF3 cells by FACS analysis. When possible dead or moribund animals in the survival study are dissected to confirm the cause of death. Morphologic and histological analyses of spleen, liver and bone marrow can also be done on these animals. Bone marrow, spleen and liver are fixed in 10% neutral buffered formalin, then processed in an automated tissue processor (TissueTek, CA) and embedded in paraffin. Four-micron thick sections are stained with H&E (Sigma, MO), and analyzed histologically for presence of infiltrating tumor cells. Photographs of histology are taken on a Nikon 80i compound microscope with a Nikon DS-R camera. All animal studies are performed under Genentech's Institutional Animal Care and Use Committee (IACUC) approved protocols.

Statistical Analyses

Error bars where presented represent mean±SEM. Student's t-test (two tailed) was used for statistical analyses to compare treatment groups using GraphPad Prism 5.00 (GraphPad Software, San Diego, Calif.). A P-value <0.05 was considered statistically significant (*p<0.05, **p<0.01, ***p<0.001 and ****p<0.0001). For Kaplan-Meier Method of survival analysis, log-rank statistics were used to test for difference in survival.

Results

Identification of ErbB2 Mutations

The Her2 mutants identified in Table 1 were those observed in tumors from patients and/or those in the proximity of the observed mutations in the TM/JM domain region and adjoining segments as indicated.

TABLE 1
Her2 mutants in JM/TM and mutation
in regions adjoining Her2 JM/TM

| # | ErbB2 mutations |
|---|---|
| 1 | P593L |
| 2 | V597M |
| 3 | R599C |
| 4 | P601A |
| 5 | G603S |
| 6 | D607N |
| 7 | S609C |
| 8 | Y610C |
| 9 | Y610S |
| 10 | D618N |
| 11 | A622T |
| 12 | P625L |
| 13 | I628M |
| 14 | T631I |
| 15 | H632P |
| 16 | S633C |
| 17 | S633F |
| 18 | C634F |
| 19 | D639Y |
| 20 | K640N |
| 21 | G641S |
| 22 | A644T |
| 23 | A644V |
| 24 | A644D |
| 25 | E645K |
| 26 | Q646H |
| 27 | S649T |
| 28 | P650S |
| 29 | L651V |
| 30 | T652R |
| 31 | S653C |
| 32 | I654T |
| 33 | I655M |
| 34 | S656C |
| 35 | S656F |
| 36 | A657V |
| 37 | V658E |
| 38 | V659G |
| 39 | V659K |
| 40 | V659Y |
| 41 | V659N |
| 42 | V659D |
| 43 | V659E |
| 44 | V659H |
| 45 | G660D |
| 46 | G660R |
| 47 | G660E |
| 48 | L663P |
| 49 | V664I |
| 50 | V664F |
| 51 | V664V |
| 52 | V665-deIVVL |
| 53 | V665M |
| 54 | V665-del |
| 55 | V666I |
| 56 | G668E |
| 57 | V669A |
| 58 | G672R |
| 59 | I673F |
| 60 | L674V |
| 61 | I675M |
| 62 | R677L |
| 63 | R677Q |
| 64 | R678W |
| 65 | R678Q |
| 66 | Q679E |
| 67 | R680D |
| 68 | R683Q |
| 69 | R683W |
| 70 | Y685H |
| 71 | T686A |
| 72 | R688W |
| 73 | R689K |
| 74 | E693K |
| 75 | T694M |
| 76 | T694S |
| 77 | V697L |
| 78 | P699-del |
| 79 | P702L |
| 80 | G704E |
| 81 | Q709L |
| 82 | A710V |
| 83 | Q711H |
| 84 | E717K |
| 85 | E717D |
| 86 | E717Q |
| 87 | T718R |
| 88 | T718M |
| 89 | E719M |
| 90 | E719D |
| 91 | E719Q |

ErbB2 Mutants Promote IL3-Independent Cell Survival and Transformation

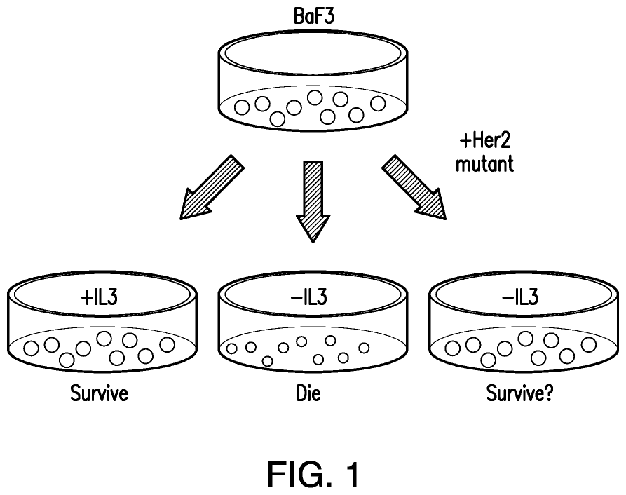

In order to further confirm the oncogenic relevance of the ErbB2 mutations we tested cell survival signaling by Her2 mutants expressed in a BaF3 system in the presence and absence of wild-type Her2 (FIG. 1). BaF3 is an interleukin (IL)-3 dependent pro-B cell line that has been widely used to study oncogenic activity of genes and development of drugs that target oncogenic drivers (Lee et al. (2006). PLoS medicine 3, e485; Warmuth et al. (2007) Current opinion in oncology 19, 55-60). Oncogenic mutants when expressed in BaF3 have been shown to substitute for IL-3 (Lee et al., 2006; Warmuth et al., 2007), thus rendering the BaF3 cells IL-3 independent by expression of an oncogene.

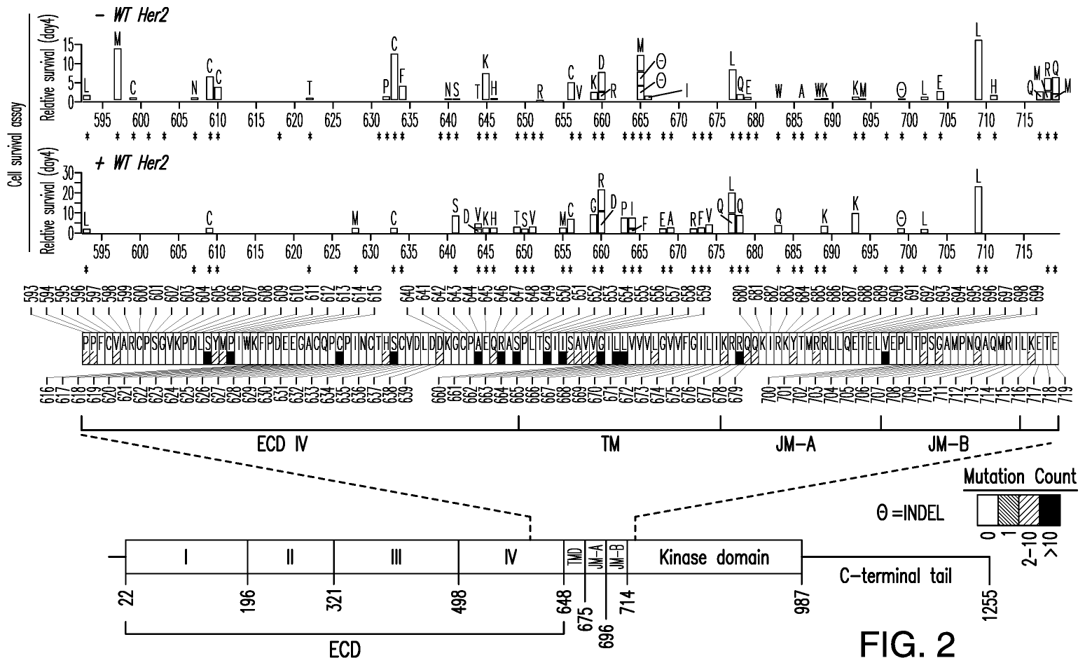

The Her2 mutants were generated based on observed mutations in tumors in patients and/or those in the proximity of the observed mutations in the TM/JM domain region and adjoining segments as indicated (Table 1). The mutations tested covered Pro 593 to Glu 719 of Her2 (FIG. 2). It includes the TM domain (Ser 649 to Ile 675) and JM domain (Val 676 to Ile 714). As depicted therein positions where mutant clones were tested are indicated by a * above the amino acid sequence number. The activating mutations in the TM, JM and adjoining regions were identified and the height of the bar show the activity of the mutants tested (i.e. the taller the bar the more active is the mutant). The ErbB2 residues and the residue number is shown below the bar graph. The background color of the residue corresponds to number of mutants observed in independent tumors as indicated in the legend. A domain diagram of ErbB2 with domain boundaries (numbered) is show at the bottom of the figure. We found multiple mutations in the TM, JM domain and the regions adjacent to JM/TM domains to be activating.

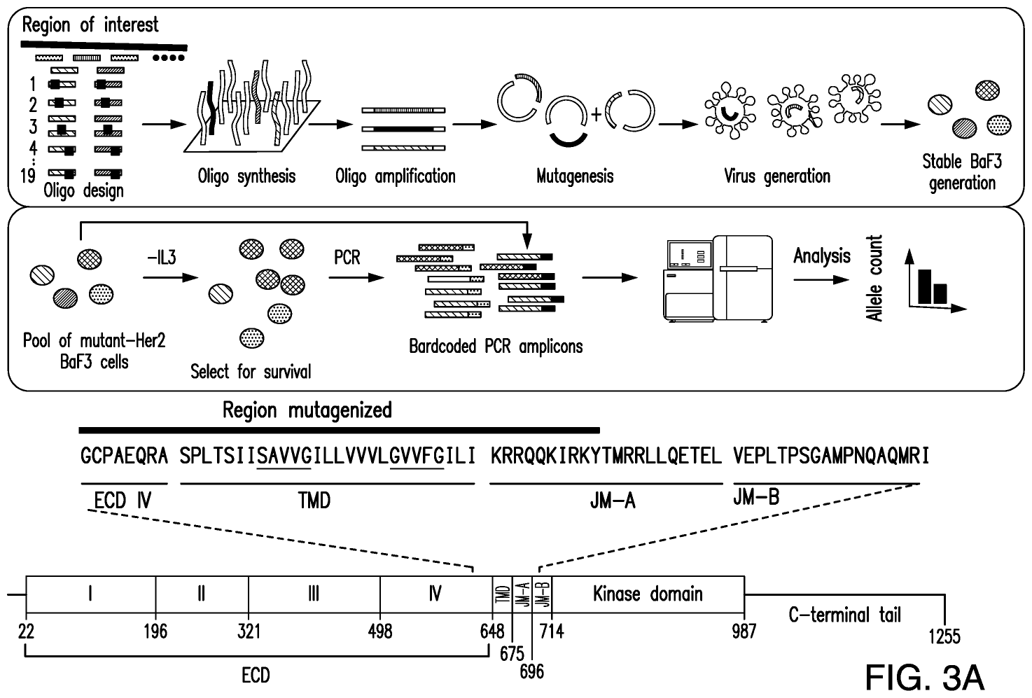

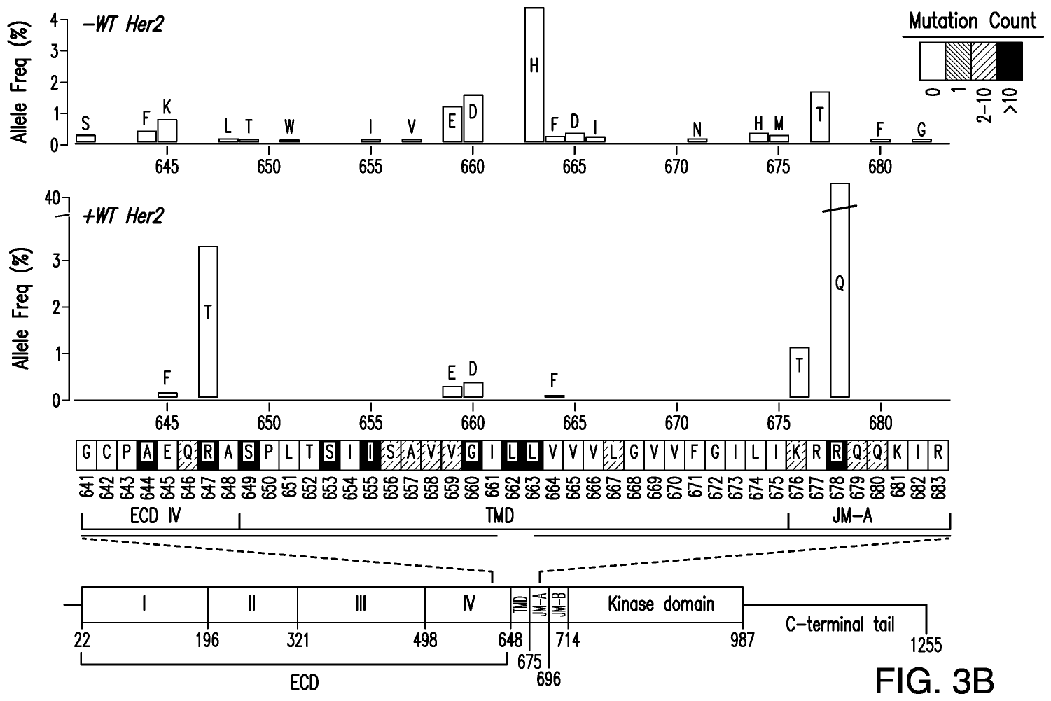

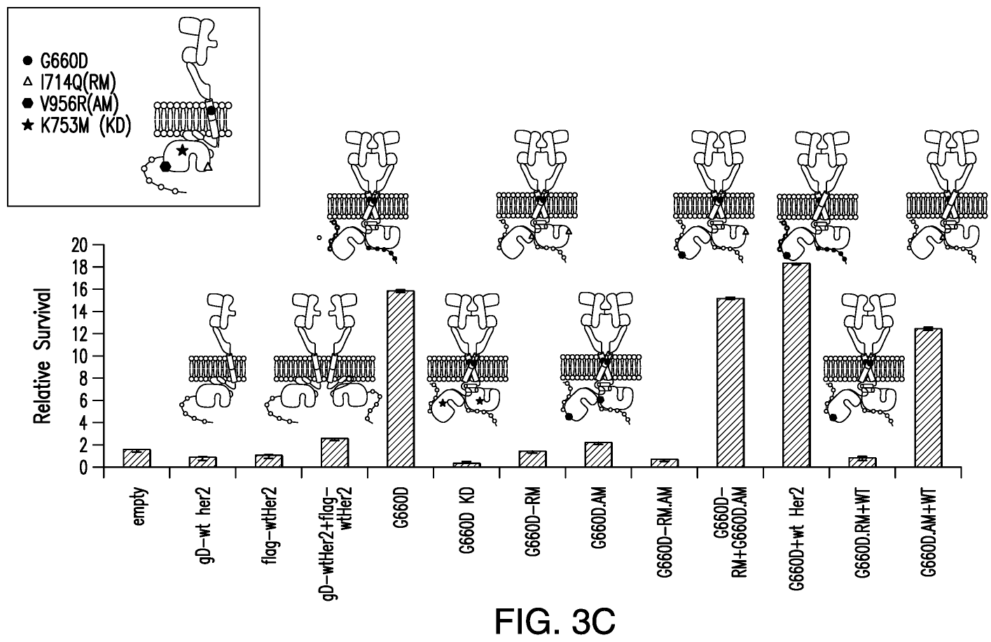

A schematic showing the workflow for the mutagenesis screen is depicted in FIG. 3A, and a bar plot representing allele frequency of HER2 mutations identified in the screen on day 4 following IL-3 removal is shown in FIG. 3B. The screen was done in the absence (FIG. 3B; upper panel) and presence (FIG. 3B; lower panel) of WT HER2, and the count of HER2 mutations observed in cancer patients is represented as color coded boxes at the bottom of FIG. 3B. Among the alleles enriched were mutants that coded for G660D and V659E. Additionally, G641S, A644F, E645K, A648L, S649T, L663H, V655D, F671N, L674H, I675M, R677T, Q680F and 1602G all showed an increase in allele frequency. In the presence of WT HER2, we found R678Q to be highly enriched followed by R647T. In addition, we found E645F, V659E, G660D and K675T to be enriched.

We also sought to understand whether HER2 mutant-signaling employed an allosteric mode of activation for its kinase domains. HER2 G660D activation involves asymmetric kinase domain dimerization and it requires a functional kinase domain for constitutive survival signaling. To test for allosteric activation, we stably expressed a kinase impaired K753M/G660D double mutant HER2 in BaF3 cells and assessed it for survival signaling in the absence of IL-3 (FIG. 3C). HER2 G660D activation involves asymmetric kinase domain dimerization and it requires a functional kinase domain for constitutive survival signaling. Compared to G660D HER2, the K753M/G660D double mutant did not support IL-3 independent survival of BaF3 cells, indicating that the kinase activity of G660D is essential for its oncogenic activity. Structure guide point mutations in the receiver or activator interface of the kinase domains have been used to confirm the role of the asymmetric dimers in the allosteric activation of ERBB kinases. We stably expressed HER2 G660D that also carried a receiver I714Q (RM) or activator impairing V956R (AM) mutation, either alone or together or in combination with WT HER2 in BaF3 cells and assayed for survival activity. Expression of receiver impaired HER2 G660D-1714Q (RM) or activator impaired HER2 G660D-V956R (AM) on its own did not promote BaF3 cell survival following IL-3 withdrawal. However, combined expression of HER2 G660D-I714Q (RM) and HER2 G660D-V956R (AM) in BaF3 cells restored the cell survival signaling activity of HER2 G660D confirming that the allosteric activation of the kinase domain following HER2 G660D dimerization promotes cell survival signaling. Since, HER2 G660D mutant can promote survival signaling in the presence of WT HER2 in BaF3 cells we tested if it preferentially functioned as a receiver or activator in the presence of WT HER2. While expression of HER2 G660D-1714Q (RM) in BaF3 cells in the presence of WT HER2 did not promote cell survival, revealing that it was not able to function as an activator of WT HER2, HER2 G660D-V956R (AM) promoted cell survival in the presence of WT HER2 indicating that the HER2 G660D is predisposed to adopt a receive confirmation.

Targeted Therapeutics are Effective Against ErbB2 Mutants

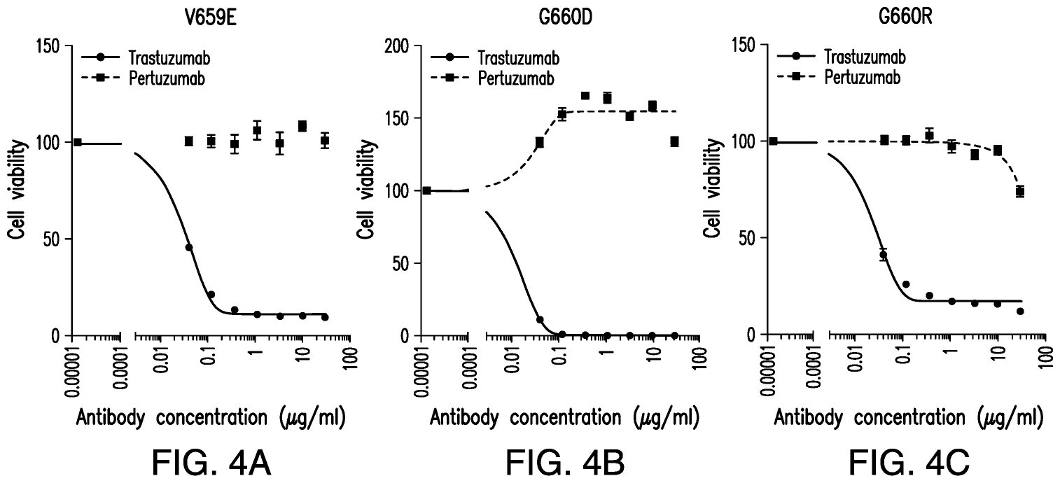

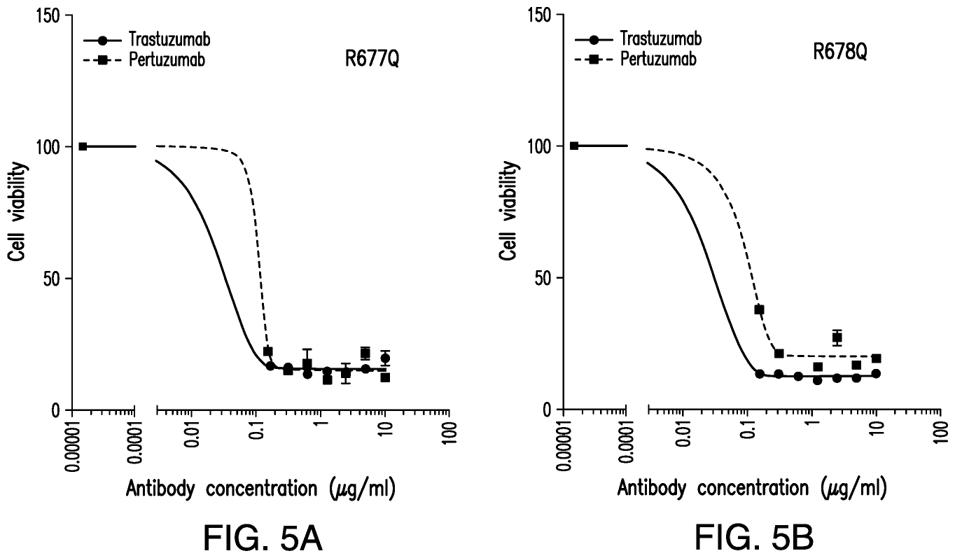

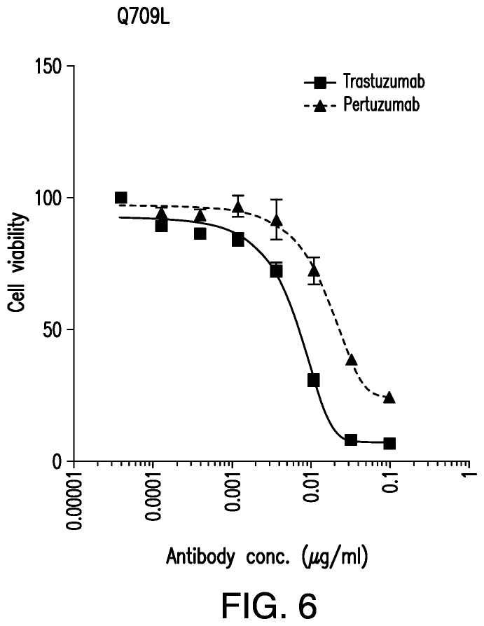

Multiple agents that target the ErbB receptors directly are approved for treating various cancers (Baselga and Swain Nature Reviews Cancer 9, 463-475 (2009); Alvarez et al. Journal of Clinical Oncology 28, 3366-3379 (2010)). Several additional candidate drugs that target ErbB family members, including ErbB2, and their downstream components are in various stages of clinical testing and development (Alvarez et al. Journal of Clinical Oncology 28, 3366-3379 (2010)). We tested trastuzumab—an anti-ErbB2 antibody that binds ErbB2 domain IV (Junttila et al. Cancer Cell 15, 429-440 (2009)) and pertuzumab—an anti-ErbB2 antibody that binds ErbB2 domain II and prevents dimerization (Junttila et al. Cancer Cell 15, 429-440 (2009)) for their effect on cell survival using the BaF3 system (FIG. 4, FIG. 5, and FIG. 6).

We found the anti-Her2 antibodies trastuzumab and pertuzumab to be effective in blocking the activity of the TM/JM Her2 mutants. In the BaF3 cell viability assays, trastuzumab was effective against all the mutants tested. Specifically, V659E, G660D and G660R Her2 TM domain mutant mediated cell survival signaling is blocked by trastuzumab (FIG. 4). However, both trastuzumab and pertuzumab were effective in blocking the three JM domain mutants tested. Specifically, R667Q, R678Q and Q709L Her2 JM domain mutant mediated cell survival signaling (FIG. 5 and FIG. 6).

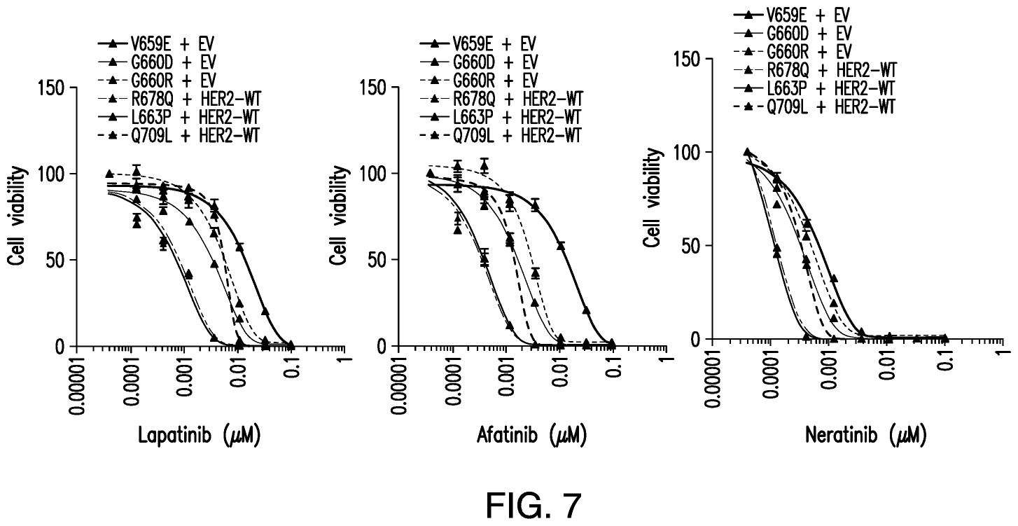

We also tested multiple ErbB2 kinase inhibitory small molecule drugs (e.g., lapatinib, afatinib and neratinib) for their effect on cell survival using the BaF3 system (FIG. 7). We found that all of the Her2 TM/JM mutants tested respond to the indicated ERBB2 kinase inhibitory small molecule drugs.

These data indicate that multiple therapeutics, either in development or approved for human use, can be effective against ErbB2-mutant driven tumors.

These functional studies demonstrate the oncogenic nature of both the TM and JM domain ErbB2 mutations. Having tested different therapeutic agents for their utility in targeting ErbB2-mutant driven oncogenic signaling, we found anti-ErbB2 antibodies to be quite effective in blocking oncogenic signaling in both TM and JM domain ErbB2 mutants. Interestingly, pertuzumab was not as effective in blocking the TM domain mutants, indicating a possibly distinct mode of action by these mutants. Previous studies have shown that while pertuzumab is quite effective in blocking ligand-mediated ErbB3/ErbB2 dimerization, trastuzumab is more effective in blocking ligand-independent ErbB2/ErbB3 dimer formation (Junttila, T. T. et al. Cancer Cell 15, 429-440 (2009)).

Assessment of ErbB2 Mutants on Promoting Oncogenesis In Vivo

We and others have shown that BaF3 cells, rendered IL-3-independent by ectopic expression of oncogenes, promote leukemia-like disease when implanted in mice and lead to reduced overall survival (Horn et al. Oncogene 27, 4096-4106 (2008); Jaiswal et al. Cancer Cell 16, 463-474 (2009)). The ability of BaF3 cells expressing ErbB2-WT, TM-mutants (V659E, G660D or G660R) or the JM domain ErbB2-mutants (R667Q and R678Q) may be tested for their ability to promote leukemia-like disease. BaF3 cells transduced with ErbB3-WT alone or ErbB2 together with empty vector may be used as controls. Mice transplanted with BaF3 cells expressing ErbB2 mutants are then assessed for median survival and development of leukemia like disease. To follow disease progression necropsies are conducted at 20 days on an additional cohort of three mice per treatment. Bone marrow, spleen, and liver samples from these animals are reviewed for pathological abnormalities. As the BaF3 cells are tagged with eGFP, we can examine isolated bone marrow and spleen for infiltrating cells by fluorescence-activated cell sorting (FACS). Consistent with a decreased survival, bone marrow and spleen from mice transplanted with cells expressing ErbB2 mutants will show a significant proportion of infiltrating eGFP-positive cells compared with bone marrow and spleen from mice receiving ErbB2-WT or empty-vector control cells. Further, concordant with a longer latency observed in ErbB2-WT cells, a very low level of infiltrating eGFP positive cells will likely be detected in the liver and spleen from these animals. Also, animals from the ErbB2 mutant arm will be expected to show increased spleen and liver size and weight compared to empty vector control or ErbB2-WT at 20 days, further confirming the presence of infiltration cells. Additionally, histological evaluation of hematoxylin and eosin (H&E) stained bone marrow, spleen and liver sections may show significant infiltration of blasts in animals with cells expressing ErbB2-mutants when compared to control at day 20. These results will demonstrate the in vivo oncogenic potential of the ErbB2 mutants.

### REFERENCES

Bose, R., Kavuri, S. M., Searleman, A. C., Shen, W., Shen, D., Koboldt, D. C., Monsey, J., Goel, N., Aronson, A. B., Li, S., et al. (2013). Activating Her2 mutations in Her2 gene amplification negative breast cancer. Cancer Discov 3, 224-237.
Chmielecki, J., Ross, J. S., Wang, K., Frampton, G. M., Palmer, G. A., Ali, S. M., Palma, N., Morosini, D., Miller, V. A., Yelensky, R., et al. (2015). Oncogenic alterations in ErbB2/Her2 represent potential therapeutic targets across tumors from diverse anatomic sites of origin. Oncologist 20, 7-12.
Greulich, H., Kaplan, B., Mertins, P., Chen, T. H., Tanaka, K. E., Yun, C. H., Zhang, X., Lee, S. H., Cho, J Ambrogio, L., et al. (2012). Functional analysis of receptor tyrosine kinase mutations in lung cancer identifies oncogenic extracellular domain mutations of ErbB2. Proc Natl Acad Sci USA 109, 14476-14481.
Jaiswal, B. S., Janakiraman, V., Kljavin, N. M., Chaudhuri, S., Stern, H. M., Wang, W., Kan, Z., Dbouk, H. A., Peters, B. A., Waring, P., et al. (2009). Somatic Mutations in p85a Promote Tumorigenesis through Class IA PI3K Activation. Cancer Cell 16, 463-474.
Jaiswal, B. S., Kljavin, N. M., Stawiski, E., Chan, E., Parikh, C., Durinck, S., Chaudhuri, S., Pujara, K., Guilory, J., Edgar, K., A., et al. (2011). Oncogenic ErbB3 mutations in human cancers. Submitted.
Ou, S. I., Schrock, A. B., Bocharov, E. V., Klempner, S. J., Haddad, C. K., Steinecker, G., Johnson, M., Gitlitz, B. J., Chung, J., Campregher, P. V., et al. (2017). Her2 Transmembrane Domain (TMD) Mutations (V659/G660) That Stabilize Homo- and Heterodimerization Are Rare Oncogenic Drivers in Lung Adenocarcinoma That Respond to Afatinib. J Thorac Oncol 12, 446-457.
Wang, S. E., Narasanna, A., Perez-Tones, M., Xiang, B., Wu, F. Y., Yang, S., Carpenter, G., Gazdar, A. F., Muthuswamy, S. K., and Arteaga, C. L. (2006). Her2 kinase domain mutation results in constitutive phosphorylation and activation of Her2 and EGFR and resistance to EGFR tyrosine kinase inhibitors. Cancer Cell 10, 25-38.
Yamamoto, H., Higasa, K., Sakaguchi, M., Shien, K., Soh, J., Ichimura, K., Furukawa, M., Hashida, S., Tsukuda, K., Takigawa, N., et al. (2014). Novel germline mutation in the transmembrane domain of Her2 in familial lung adenocarcinomas. J Natl Cancer Inst 106, djt338.

1-69. (canceled)

70. A method of treating cancer in a human subject in need comprising
a) detecting in a biological sample obtained from the subject the presence or absence of an ErbB2 somatic mutation in a nucleic acid sequence encoding ErbB2, wherein the mutation results in an amino acid variation at at least one position within the transmembrane (TM) or juxtamembrane (JM) domain of a native human ErbB2 amino acid sequence and wherein the mutation is indicative of a cancer in the subject; and
b) administering an anti-cancer therapeutic agent to said subject.

71. The method of claim 70, wherein the mutation is an activating ErbB2 somatic mutation.

72. The method of claim 70, wherein the mutation resulting in an amino acid change is at a position of ErbB2 selected from the group of mutations listed in Table 1.

73. The method of claim 70, wherein the therapeutic agent is an ErbB2 antagonist.

74. The method of claim 73, wherein the ErbB2 antagonist is a small molecule inhibitor, an antagonist anti-ErbB2 antibody or an anti-ErbB2 antibody-drug conjugate.

75. The method of claim 74, wherein the small molecule inhibitor is an ErbB2 kinase inhibitor.

76. The method of claim 75, wherein the ErbB2 kinase inhibitor is selected from the group consisting of lapatinib, afatinib and neratinib.

77. The method of claim 74, wherein the anti-ErbB2 antibody is trastuzumab or pertuzumab.

78. The method of claim 74, wherein the ErbB2 antagonist is trastuzumab-MCC-DM1 (T-DM1, trastuzumab emtansine).

79. The method of claim 70, wherein the cancer is selected from the group consisting of breast, gastric, colon, esophageal, rectal, cecum, colorectal, biliary, urothelial, bladder, salivary, non-small-cell lung (NSCLC) adenocarcinoma, NSCLC (Squamous carcinoma), renal carcinoma, melanoma, ovarian, lung large cell, small-cell lung cancer (SCLC), hepatocellular (HCC), lung, and pancreatic cancer.

80. A method of determining the efficacy of an ErbB2 blocking antibody or antibody-drug conjugate, comprising
a) detecting in a biological sample obtained from a subject treated with an ErbB2 blocking antibody a mutation in a nucleic acid sequence encoding ErbB2, wherein the mutation results in an amino acid variation at least one position within the transmembrane (TM) or juxtamembrane (JM) domain of a native human ErbB2 amino acid sequence and wherein the mutation is indicative of an ErbB2 mutated cancer in the subject; and
b) predicting a therapeutic response in said subject based on the ErbB2 mutation detected.

81. The method of claim 80, wherein the mutation resulting in an amino acid change is at a position of ErbB2 selected from the group of mutations listed in Table 1.

82. The method of claim 80, wherein the mutation is the mutation is a Her2-activating mutation.

83. The method of claim 80, wherein the antibody is selected from the group consisting of a monoclonal antibody, a bispecific antibody, a chimeric antibody, a human antibody, a humanized antibody and an antibody fragment.

84. The method of claim 80, wherein the antibody or antibody-drug conjugate is trastuzumab, trastuzumab-MCC-DM1 (T-DM1) or pertuzumab.

85. The method of claim 80, wherein the ErbB2 mutated cancer is selected from the group consisting of breast, gastric, colon, esophageal, rectal, cecum, colorectal, biliary, urothelial, bladder, salivary, non-small-cell lung (NSCLC) adenocarcinoma, NSCLC (Squamous carcinoma), renal carcinoma, melanoma, ovarian, lung large cell, small-cell lung cancer (SCLC), hepatocellular (HCC), lung, and pancreatic.

86. A method for determining whether a patient is expected to be responsive to anti-ErbB2 therapy, comprising the steps of:
a) obtaining a sample of cellular material from a human subject;
b) examining nucleic acid material from at least part of one or more ErbB2 genes in said cellular material; and
c) determining whether such nucleic acid material comprises one or more mutations in a sequence encoding the transmembrane (TM) or juxtamembrane (JM) domain of a native human ErbB2 polypeptide,
wherein the presence of one or more mutations is indicative that the patient is expected to be responsive to anti-ErbB2 therapy.

87. The method of claim 86, wherein the mutation is selected from the group of mutations listed in Table 1.

88. The method of claim 86, where the anti-ErbB2 therapy is an antagonist anti-ErbB2 antibody or an anti-ErbB2 antibody-drug conjugate.

89. The method of claim 86, wherein the ErbB2-positive cancer is selected from the group consisting of breast, gastric, colon, esophageal, rectal, cecum, colorectal, biliary, urothelial, bladder, salivary, non-small-cell lung (NSCLC) adenocarcinoma, NSCLC (Squamous carcinoma), renal carcinoma, melanoma, ovarian, lung large cell, small-cell lung cancer (SCLC), hepatocellular (HCC), lung, and pancreatic.


SEQUENCE LISTING (4 sequences)

SEQ ID NO: 1 - DNA, 4473 bp, Homo sapiens
aaggggaggtaaccctggcccctttggtcggggccccgggcagccgcgcgccccttcccacggggccctttactgcgccgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggag

SEQ ID NO: 2 - protein, 1255 aa, Homo sapiens [sequence omitted]

SEQ ID NO: 3 - protein, 74 aa, Artificial Sequence
  Description of Artificial Sequence Synthetic polypeptide
GCPAEQRASPLTSIISAVVGILLVVVLGVVFGILIKRRQQKIRKYTMRRLLQETELVEPLTPSGAMPNQAQMRI

SEQ ID NO: 4 - protein, 43 aa, Artificial Sequence
  Description of Artificial Sequence Synthetic polypeptide
GCPAEQRASPLTSIISAVVGILLVVVLGVVFGILIKRRQQKIR